(19) United States
(12) Patent Application Publication
(10) Pub. No.: US 2019/0209724 A1
(43) Pub. Date: Jul. 11, 2019

(54) DECONTAMINATION DEVICE FOR PHARMACEUTICAL VESSELS

(71) Applicant: Mikael Naygauz, Forrest Hills, NY (US)

(72) Inventor: Mikael Naygauz, Forrest Hills, NY (US)

(21) Appl. No.: 16/358,434

(22) Filed: Mar. 19, 2019

Related U.S. Application Data
(63) Continuation-in-part of application No. 16/100,964, filed on Aug. 10, 2018.
(60) Provisional application No. 62/562,490, filed on Sep. 25, 2017.
(60) Provisional application No. 62/612,267, filed on Dec. 29, 2017.
(60) Provisional application No. 62/582,922, filed on Nov. 7, 2017.
(60) Provisional application No. 62/560,195, filed on Sep. 19, 2017.
(60) Provisional application No. 62/558,502, filed on Sep. 14, 2017.
(60) Provisional application No. 62/631,729, filed on Feb. 17, 2018.
(60) Provisional application No. 62/625,334, filed on Feb. 1, 2018.
(60) Provisional application No. 62/626,686, filed on Feb. 6, 2018.
(60) Provisional application No. 62/651,000, filed on Mar. 30, 2018.
(60) Provisional application No. 62/651,149, filed on Mar. 31, 2018.
(60) Provisional application No. 62/653,356, filed on Apr. 5, 2018.
(60) Provisional application No. 62/657,808, filed on Apr. 15, 2018.
(60) Provisional application No. 62/679,817, filed on Jun. 3, 2018.
(60) Provisional application No. 62/680,576, filed on Jun. 4, 2018.

(51) Int. Cl. A61L 2/232; A61L 2/02

(52) U.S. Cl. CPC A61L 2/232; A61L 2202/23; A61L 2202/15; A61L 2/02

(57) ABSTRACT

The present invention relates, in some embodiments thereof, to systems, devices and methods for decontaminating a surface of one or more vessels. In some embodiments, the systems and devices of the invention include a housing, and a wiping member disposed within the housing being configured to wipe off the surface of one or more vessels, thereby establishing a contaminant-free fluid passageway between vessels.

### CROSS REFERENCE TO RELATED APPLICATIONS

This application is a continuation-in-part application of U.S. application Ser. No. 16/100,964 filed on Aug. 10, 2018, which claims priority to 62/558,502 filed on Sep. 14, 2017; 62/560,195 filed on Sep. 19, 2017; 62/562,490 filed on Sep. 25, 2017; 62/582,922 filed on Nov. 7, 2017; 62/612,267 filed on Dec. 29, 2017; 62/625,334 filed on Feb. 1, 2018; 62/626,686 filed on Feb. 6, 2018; 62/631,729 filed on Feb. 17, 2018; 62/651,000 filed on Mar. 30, 2018; 62/651,149 filed on Mar. 31, 2018; 62/653,356 filed on Apr. 5, 2018; 62/657,808 filed on Apr. 15, 2018; 62/679,817 filed on Jun. 3, 2018; 62/680,576 filed on Jun. 4, 2018. The contents of the above applications are all incorporated by reference as if fully set forth herein in their entirety.

### FIELD OF THE INVENTION

The present invention relates, in some embodiments thereof, to systems and methods for decontaminating a surface of one or more vessels. In some embodiments, the systems and methods of the invention include a housing, and a wiping member disposed within the housing and configured to wipe off the surface of one or more vessels, thereby establishing a contaminant-free fluid passageway between vessels.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces and consequently contamination of a beneficial substance to be provided to a patient.

Existing systems include U.S. D720067; U.S. D717947; U.S. D703812; U.S. D690418; U.S. D639939; U.S. D637713; U.S. Pat. Nos. 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Thus, there is a long felt and unmet need for pharmaceutical vessels, devices, systems and/or methods that afford transfer of medical substances in a sterile manner. There is a need for reliable, user friendly and cost-effective solutions allowing contaminant-free engagement of vessels and devices for drug preparation and administration processes.

### SUMMARY OF THE INVENTION

Objects of the invention are achieved by providing systems, devices and methods for administering medical substances in a decontaminated manner.

Objects of the invention are achieved by providing systems, devices and methods which are directed to transferring medical substances in an efficient, user-friendly and sterile manner.

The present invention provides devices and systems that decontaminate connection surfaces of medical vessels, pharmaceutical vessels or pharmaceutical administration devices and thereafter allow decontaminated fluid passageway between the vessels.

In a first aspect, the present invention provides a device for decontaminating a surface of at least one vessel, comprising: a housing; and a wiping member disposed within the housing, wherein the wiping member is configured to move within the housing; wherein the wiping member decontaminates the surface of the at least one vessel.

In one or more embodiments, the device further comprising an actuator disposed through a wall of the housing, the actuator configured to move the wiping member.

In one or more embodiments, the device further comprising an actuator disposed in a wall of the housing, the actuator configured to move the wiping member.

In a further aspect, the invention provides a device for decontaminating a surface of at least one vessel, the device comprising:
a housing configured to allow an engagement with the at least one vessel;
a wiping member disposed within the housing; and
a wiping member actuator configured to maneuver movement of the wiping member along the housing upon a pushing motion exerted by the engagement of the at least one vessel with the housing, wherein the movement of the wiping member along the housing decontaminates the surface of the at least one vessel.

In one or more embodiments, the system further comprises an actuator configured to move the wiping member within the housing. In one or more embodiments, the actuator is selected from a group consisting of a handle, tab, a button, a touch button, a lever, a gear, a spring, a mechanical actuator, an electric actuator, and combinations thereof. In one or more embodiments, the actuator is selected from a group consisting of a lever, a gear, a spring, a mechanical actuator, an electrical actuator, a string, a clamp, a pin and combinations thereof. In one or more embodiments, the actuator is connected to the wiping member and configured to move the wiping member. In one or more embodiments, the actuator is connected to the wiping member and configured to forcibly move the wiping member when the at least one vessel engages the housing. In one or more embodiments, the actuator moves the wiping member via a pushing, pulling and/or sliding motion. In one or more embodiments, the actuator manipulates the wiping member from the exterior of the housing. In one or more embodiments, the actuator is connected to the wiping member and configured to move the wiping member across the housing. In one or more embodiments, the actuator is spring loaded. In one or more embodiments, the actuator extends through an opening in a wall of the housing. In one or more embodiments, the actuator extends through an opening in a wall of the housing, wherein an airtight seal is maintained between the actuator and the opening in the wall. In one or more embodiments, the system may be a device In one or more embodiments, the actuator disposed within the housing, outside the housing or both. In one or more embodiments, the pushing motion exerted by the engagement of the vessel with the housing transfers a force to the actuator to thereby allow the movement of the wiping member along the housing. In one or more embodiments, the actuator holds the wiping member in an initial position and allows movement of the wiping member to an end position following pushing the vessel into a port of the housing. In one or more embodiments, the actuator is a resilient string connected at one end thereof to the wiping member and having a second end or portion that protrudes into a lumen of a vessel's port within the housing. In one or more embodiments, the string holds the wiping member in an initial position and wherein pushing a vessel into a port of the housing pushes the second end or portion of the string, thereby releasing holding of the wiping member in the initial position and allowing movement thereof to an end position.

In one or more embodiments, the system further comprises a transfer mechanism disposed in a wall of the housing, the transfer mechanism configured to transfer a force from the actuator to the wiping member to move the wiping member within the housing. In one or more embodiments, the housing has a side wall, a top wall, and a bottom wall, wherein the top wall and the bottom wall each have an opening adapted for connection to the at least one vessel. In one or more embodiments, the housing further comprises a second side wall.

In one or more embodiments, the openings have vertical walls with internal threads, the internal threads being configured for cooperating with external threads on the at least one vessel to connect the vessel to the housing. Alternately, the vertical walls have external thread, the external thread being configured for cooperating with internal threads on the at least one vessel to connect the vessel to the housing. Optionally, the openings have non-vertical wall with internal or external threads. In one or more embodiments, the openings are configured to engage with the at least one vessel via a ratchet teeth mechanism, a snap-on mechanism, a slide-on mechanism, a twist-on mechanism, an adhesive or combinations thereof.

In one or more embodiments, the housing has two wiping members disposed one above the other, wherein one of the wiping members is arranged to wipe a surface of a vessel connected to the bottom wall of the housing, and the other of the wiping members is arranged to wipe a surface of a vessel connected to a top of the housing.

In one or more embodiments, the bottom wall of the housing contains a plurality of the openings configured for connecting a plurality of vessels to the housing, and wherein the system is configured so that actuating the wiping member wipes the surfaces of a plurality or of all vessels connected to the housing.

In one or more embodiments, the side wall of the housing is open opposite the wiping member. In one or more embodiments, the wiping member is configured to extend from the top wall to the bottom wall of the housing. In one or more embodiments, the housing has a hermetic seal between the top wall and the bottom wall of the housing during actuation of the wiping member. In one or more embodiments, the wiping member forms a hermetic seal between the top wall and the bottom wall of the housing.

In one or more embodiments, the system further comprises a filter disposed in an aperture in the housing for releasing air or air pressure from the housing when actuating the wiping member. In one or more embodiments, the filter is less than 0.5 microns porosity. In one or more embodiments, the filter is a 0.22 micron or less porosity filter. In one or more embodiments, the filter is a sterilizing filter. In one or more embodiments, the system further comprises a valve disposed in an aperture of the housing for releasing air or air pressure from the housing while preventing ambient or environmental air from entering the housing. In one or more embodiments, the valve is a one-way valve.

In one or more embodiments, the system further comprises a locking member disposed in or on the housing, the locking member being configured for locking the wiping member in an end position after actuation of the actuator to prevent the wiping member from returning or retracting to an initial position.

In one or more embodiments, the at least one vessel ranges from one vessel to ten vessels, wherein a surface of each of the vessels is configured to be wiped off by the wiping member, wherein each of the vessels are configured to couple with the housing.

In one or more embodiments, the housing is manufactured from a group consisting of plastic, rigid plastic, flexible plastic, polymer, glass and combinations thereof.

In one or more embodiments, the wiping member is comprised of an elastomeric material and/or a rubber material. In one or more embodiments, the wiping member is comprised of a rigid material, flexible material, expandable material, absorptive material, and combinations thereof. Other types of wiping members disposed within or at least partially disposed within the housing are contemplated such as wiping members that rotate on an axle, hinge, spindle, axis, and/or rod. Rotation of at least one of the wiping members may be less than 360 degrees, less than 180 degrees or less than 90 degrees. In one or more embodiments, the wiping member is covered by a sterilizing substance and/or disinfecting substance. In one or more embodiments, the sterilizing substance is an antibacterial, an antiviral, an anti-tuberculin, an anti-fungal, an anti-HIV and combinations thereof. In one or more embodiments, the system is disposable, intended for one-time use, or repeat use. In one or more embodiments, the wiping member is configured to move across the housing via a sliding motion. In one or more embodiments, the wiping member is configured to partially wipe off the surface of at least one vessel. In one or more embodiments, the wiping member is configured to wipe off the entire surface of at least one vessel. In one or more embodiments, the wiping member is configured to wipe off the portion of a surface of a vessel where a fluidic communication/transfer occurs. In one or more embodiments, the system includes a plurality of vessels.

In one or more embodiments, the wiping member is configured to partially wipe off a surface of each of the plurality of vessels. In one or more embodiments, the wiping member is configured to entirely wipe off an entire surface of each of the plurality of vessels. In one or more embodiments, the wiping member is configured to wipe off a portion of the surfaces of vessels where a fluidic communication/transfer occurs. In one or more embodiments, the wiping member is configured to wipe off the surface of the plurality of vessels at about the same time or at the same time. In one or more embodiments, the wiping member is configured to displace at least one cover covering the surface of the at least one vessel. In one or more embodiments, the wiping member is configured to displace at least one seal of the at least one vessel. In one or more embodiments, the vessel has a displaceable cover or seal. In one or more embodiments the vessel has a displaceable cover or seal that snaps off, breaks off, or tears off when the wiping member moves across the vessel surface. In one or more embodiments the displaceable cover or seal is secured to the vessel surface via a frangible seal. In one or more embodiments, the displaceable cover and/or seal is disposed on a rail mechanism, wherein the rail mechanism is disposed and/or attached to a vessel. In one or more embodiments, the rail mechanism is integrally attached to the vessel. In one or more embodiments, the displaceable cover and/or seal is integrally attached to the vessel. In one or more embodiments, the rail mechanism forms a unitary structure with the vessel. In one or more embodiments, the displaceable cover and/or seal of the vessel displaces via a hinge mechanism, wherein the hinge mechanism is attached, or optionally integrally attached to the vessel.

In one or more embodiments, the housing has an engagement mechanism to connect the at least one vessel to the housing. In one or more embodiments, the engagement mechanism is selected from a thread, a luer, a luer-lock, a luer-slip, a ratchet teeth mechanism, a retention member, a rail mechanism, a clamping rail mechanism, an adhesive mechanism, and combinations thereof. In one or more embodiments, the thread is configured for engaging a complementary thread on the at least one vessel to connect the at least one vessel to the housing. In one or more embodiments, the thread and the complementary thread may be a luer. In one or more embodiments, the engagement mechanism positions the surface of the at least one vessel at about same level as an interior wall of the housing. In one or more embodiments, the engagement mechanism positions the surface of the pharmaceutical vessel at the same level as an interior wall of a housing of the system. In one or more embodiments, the engagement mechanism positions the surface of the at least one vessel in a manner that protrudes into the housing. In one or more embodiments, the protruding of the surface of the at least one vessel is a slight/minor protruding. In one or more embodiments, the protruding of the surface of the at least one vessel is ranges from less than one eighth of an inch to less than half an inch. In one or more embodiments, the engagement mechanism positions the surface of the at least one vessel slightly above an inner wall/surface of the housing while allowing the wiping member to wipe off the entire surface of the at least one vessel. In one or more embodiments, the engagement mechanism positions the surface of the at least one vessel in a manner that is below an inner surface of the housing. In one or more embodiments, the positioning of the surface of the at least one vessel below an inner surface of the housing ranges from less than one eighth of an inch to less than a half an inch from the inner surface of the housing. In one or more embodiments, the engagement mechanism positions the surface of the at least one vessel slightly below an inner wall/surface of the housing while allowing the wiping member to wipe off the entire surface of the at least one vessel.

In one or more embodiments, the system further comprising at least one port configured for coupling to the at least one vessel, wherein the port provides a connection between the housing and the at least one vessel. In one or more embodiments, the port is covered by a cover. In one or more embodiments, the port is sealed. In one or more embodiments, the port is sealed by a frangible seal. In one or more embodiments, the port is configured to attach or engage a connector, wherein the connector is configured to attach to the port and to a vessel. In one or more embodiments, the system comprises two or more ports each configured to attach to a plurality of connectors, wherein the connectors are configured to attach to the ports of the housing and to a corresponding vessel.

In one or more embodiments, the port has an adhesive configured for engaging the at least one vessel to connect the at least one vessel to the housing. In one or more embodiments, the bottom wall of the housing contains a plurality of ports configured for connecting a plurality of vessels to the housing, wherein the housing is configured so that actuating the wiping member wipes the surfaces of the plurality of vessels connected to the housing. In one or more embodiments, the housing is comprised of a top wall and a bottom wall, wherein the top wall and the bottom wall each have at least one port configured for connection to at least one vessel.

In one or more embodiments, the top and bottom ports are disposed one above the other. In one or more embodiments, when each of the ports is connected to a vessel, the housing forms an interior which is hermetically sealed/isolated from surrounding/environmental air. In one or more embodiments, the housing includes a plurality of ports configured for connection to a plurality of vessels. In one or more embodiments, each of the plurality of ports provides airtight engagement between the housing and a vessel. In one or more embodiments, the housing has at least two ports, wherein one of the at least two ports are attached to or integrally attached to the at least one vessel. In one or more embodiments the housing has at least two ports, wherein one of the at least two ports forms a unitary structure with the at least one vessel. In one or more embodiments, the housing has at least two ports, wherein one of the at least two ports are permanently/fixedly attached to the at least one vessel. In one or more embodiments, the housing has at least two ports, wherein one of the at least two ports are attached to or coupled to the at least one vessel at about the time of manufacture, assembly and/or distribution of the system or device. In one or more embodiments, the port is a container port, spike port, syringe port, infusion line port, connector port, manifold port, a vessel port, and combinations thereof. In one or more embodiments, the at least one vessel is a container, a bag, a bottle, a syringe, an infusion line, a connector, a filter, a manifold, a bag port, a bottle port, a vial port, and combinations thereof. In one or more embodiments, the surface of the at least one vessel is flat and smooth. In one or more embodiments, the contact between the wiping member and the vessel surface provides enough friction between the vessel surface and the wiping member to wipe off contaminants from the surface of the at least one vessel. In one or more embodiments, the surface of the wiping member is smooth. In one or more embodiments, the surface of the wiping member is rigid.

In yet a further aspect the present invention provides a decontamination system, comprising: a device comprising a housing, and a wiping member disposed within the housing, wherein the wiping member is configured to move within the housing; and at least one vessel having a surface, wherein the wiping member decontaminates the surface of the at least one vessel.

In one or more embodiments, the at least one vessel includes two or more vessels, wherein the wiping member is configured to wipe the surfaces of the two or more vessels. In one or more embodiments, the system further comprising a second wiping member.

In yet a further aspect the present invention provides a method of decontaminating a surface of at least one vessel, the method comprising: providing a decontamination device having a housing and a wiping member disposed within and configured to move within the housing; providing at least one vessel having a surface; connecting the decontamination device to the at least one vessel; and moving the wiping member within the housing, thus, decontaminating the surface of the at least one vessel.

In one or more embodiments, the connection of the decontamination device to the at least one vessel results in a hermetic seal being formed between the decontamination device and the at least one vessel.

In one or more embodiments, the wiping member is moved within the housing via an actuator. In one or more embodiments, the actuator is selected from a group consisting of a handle, tab, a button, a touch button, a lever, a gear, a spring, a mechanical actuator, an electric actuator, and combinations thereof. In one or more embodiments, the actuator is connected to the wiping member and configured to move the wiping member. In one or more embodiments, the actuator moves the wiping member via a pushing, pulling and/or sliding motion. In one or more embodiments, a pressurized gas within a compartment of the housing may move the wiping member across the housing. In one or more embodiments, the pressurized gas may be released via an actuator located on an exterior wall of the housing or within an aperture of a wall of the housing. In one or more embodiments, the actuator manipulates the wiping member from an exterior of the housing. In one or more embodiments, the actuator is connected to the wiping member and configured to move the wiping member across the housing. In one or more embodiments, the actuator is spring loaded. In one or more embodiments, the wiping member is moved or slid within the housing via a force of a hand of a user applied to the housing wall, wherein the housing of the wall is flexible and is configured to allow the user to manipulate the location or movement of the wiping member through a wall of the housing.

In one or more embodiments, the decontamination device is pre-attached to the at least one vessel. In one or more embodiments, the decontamination device is integrally attached to the at least one vessel. In one or more embodiments, the decontamination device forms a unitary structure with at least one vessel. In one or more embodiments, moving the wiping member across the housing occurs when or after the housing is hermetically sealed to the at least one vessel. In one or more embodiments, moving the wiping member across the housing occurs at about the time the at least one vessel is coupled or engaged to the housing.

In one or more embodiments, the decontamination device has a safety mechanism configured to prevent the movement of the wiping member prior to attachment of at least one vessel to the housing of the decontamination device. In one or more embodiments, the decontamination device has a safety mechanism configured to prevent actuation of the wiping member actuator by a user prior to attachment of at least one vessel to the housing of the decontamination device. According to these embodiments, the safety mechanism prevents the premature movement and/or actuation of the wiping member prior to an engagement of the decontamination device to at least one vessel. In one or more embodiments, the safety mechanism is configured to prevent movement and/or piercing of a vessel surface by a piercing member. According to this embodiment, the safety mechanism prevents the premature piercing of a vessel surface prior to decontamination of the vessel surface by the wiping member.

In one or more embodiments, the decontamination device has a permanent lock mechanism which engages at least one vessel permanently to the decontamination device and/or to the housing of the decontamination device. The permanent lock mechanism may be referred to as an irreversible locking mechanism. The irreversible locking mechanism may be positioned on a wall or in a wall of the housing and configured to engage a locking member on at least one vessel. Optionally, the irreversible locking mechanism may be positioned on an opening of the housing, on a port of the housing, and/or on an engagement mechanism of the housing. In one or more embodiments, the hermetically sealed connection between the housing and the at least one vessel provides for a sealed chamber/compartment within the housing of the decontamination device that is isolated from environmental/contaminated air. In one or more embodiments, moving the wiping member across the housing occurs after engaging the at least one vessel to the decontamination device. In one or more embodiments, moving the wiping member across the housing occurs at about the time of engaging the at least one vessel to the decontamination device. In one or more embodiments, engaging/connecting the at least one vessel to the decontamination device occurs prior to moving the wiping member.

In one or more embodiments, moving the wiping member across the housing forms a chamber/compartment/housing that is sterile. In one or more embodiments, moving the wiping member across the housing forms a conduit where a fluid communication may be formed between the at least one vessel and the decontamination device. In one or more embodiments, the conduit is decontaminated. In one or more embodiments, the conduit is sterile.

In one or more embodiments, the at least one vessel is connected to the decontamination device via an engagement mechanism. In one or more embodiments, the engagement mechanism is selected from a group consisting of a thread mechanism, a luer mechanism, a ratchet teeth/retention member mechanism, and an adhesive mechanism. In one or more embodiments, connecting the decontamination device to the at least one vessel is performed via an engagement mechanism. In one or more embodiments, connecting the decontamination device to the at least one vessel includes twisting of a thread onto a complementary thread, attaching ratchet teeth to a complementary retention member, and combinations thereof. In one or more embodiments, connecting the decontamination device to the at least one vessel includes a snap-on mechanism. In one or more embodiments, the engagement mechanism is attached to the decontamination device. In one or more embodiments, the engagement mechanism is integrally attached to the decontamination device. In one or more embodiments, the engagement mechanism forms a unitary structure with the decontamination device.

In one or more embodiments, the decontamination device is connected to the at least one vessel via an adhesive. In one or more embodiments, the at least one vessel is a bottle, a bag, a container, a vial, a manifold, a spike, a connector, a connector with a plurality of ports, a syringe, an infusion line, a connector, a filter, a manifold, a bag port, a bottle port, a vial port, and combinations thereof.

In yet a further aspect the present invention provides a method of decontaminating a surface of at least two vessels, the method comprising: providing a decontamination device comprising a housing and a wiping member disposed within the housing configured to move within the housing; providing a first vessel having a surface; providing a second vessel having a surface; connecting the device to the first vessel; connecting the device to the second vessel; and moving the wiping member within the housing, thus, decontaminating a surface of the first vessel, and decontaminating a surface of the second vessel.

In one or more embodiments, moving the wiping member across the housing decontaminates a surface of the first vessel and a surface of the second vessel. In one or more embodiments, the invention provides for a method of decontaminating a surface of a vessel, the method comprising: providing a decontamination device comprising a housing and a wiping member disposed within the housing configured to move within the housing, wherein a first vessel is pre-attached to the housing; providing second vessel; connecting housing to the second vessel; and moving the wiping member within the housing across the surface of second vessel thus decontaminating a surface of the second vessel. In one or more embodiments, the wiping member is disposed within the housing between the opening of the first vessel surface and the second vessel surface. In one or more embodiments, the method comprises decontaminating the surface of one vessel.

In one or more embodiments, the first vessel is connected to the decontamination device prior to the second vessel being connected to the device. In one or more embodiments, the first vessel is connected to the decontamination device after the second vessel is connected to the device. In one or more embodiments, the first vessel is connected to the decontamination device and the second vessel is connected to the decontamination device at about the same time or at the same time. In one or more embodiments, moving of the wiping member across the housing occurs after the first vessel and the second vessel are connected to the device.

In yet another aspect, a syringe tip/opening/aperture is sealed with a puncturable and/or frangible seal. The seal is configured to be wiped off by the wiping member. In one or more embodiments, the syringe tip/opening/aperture may be covered by a seal that is punctured by piercing member of decontamination device or ruptured inside decontamination device. The syringe seal may also be displaceable inside the decontamination device.

In one or more embodiments, a syringe tip/opening/aperture is sealed with a displaceable seal or cover. In one or more embodiments the displaceable seal or cover is attached to the syringe tip/opening/aperture via a frangible seal. In one or more embodiments, the displaceable seal or cover is attached to the syringe via a frangible seal. In one or more embodiments, the syringe further comprises a displaceable seal or cover disposed on a rail mechanism of the syringe or between the rails of a rail mechanism of the syringe. In one or more embodiments, the rail mechanism of the syringe is configured to provide for the displacement of the seal or cover of the syringe tip/opening/aperture. In one or more embodiments, the syringe further comprises a hinge mechanism and a displaceable seal or cover, wherein the hinge mechanism is configured to provide for the displacement of the seal or cover.

In one or more embodiments, a container and/or vial aperture is sealed with a displaceable seal or cover. In one or more embodiments the displaceable seal or cover is attached to the container and/or vial a frangible seal. In one or more embodiments, the displaceable seal or cover is attached to the container and/or vial via a frangible seal. In one or more embodiments, the container and/or vial further comprises a displaceable seal or cover disposed on a rail mechanism of the container and/or vial or between the rails of a rail mechanism of the container and/or vial. In one or more embodiments, the rail mechanism of the container and/or vial is configured to provide for the displacement of the seal or cover of the container and/or vial. In one or more embodiments, the container and/or vial further comprises a hinge mechanism and a displaceable seal or cover, wherein the hinge mechanism is configured to provide for the displacement of the seal or cover.

In yet a further aspect the present invention provides a decontamination system, comprising: a device comprising a housing, and a wiping member disposed within the housing, wherein the wiping member is configured to move within the housing; and at least one vessel having a surface, wherein the wiping member decontaminates the surface of the at least one vessel, and wherein the at least one vessel is configured to detach from the device after the surface of the at least one vessel has been decontaminated.

In one or more embodiments, the system includes an actuator configured to move the wiping member within the housing. In one or more embodiments, the actuator is selected from a group consisting of a handle, tab, a button, a touch button, a lever, a gear, a spring, a mechanical actuator, an electric actuator, and combinations thereof. In one or more embodiments, the actuator is connected to the wiping member and configured to move the wiping member.

In one or more embodiments, the vessel includes a fluid, wherein the fluid is configured to travel through the device after the surface of the at least one vessel has been decontaminated.

In one or more embodiments, the system includes a capping or resealing mechanism that is configured to seal the surface of the at least one vessel.

In yet a further aspect the present invention provides a decontamination system, comprising: a device comprising a housing, and a wiping member disposed within the housing, wherein the wiping member is configured to move within the housing; and at least one vessel having a surface, wherein the wiping member decontaminates the surface of the at least one vessel, and a capping or resealing mechanism that is configured to seal the surface of the at least one vessel after the surface of the at least one vessel has been decontaminated.

In one or more embodiments, the resealing mechanism includes a compartment that is disposed on one end of the housing.

In one or more embodiments, the compartment includes one or more caps. In one or more embodiments, the wiping member is configured to attach to one or more of the caps and slide there along housing when attached thereto. In one or more embodiments, the wiping member reseals the at least one vessel with the caps. In one or more embodiments, the caps are disposed within the housing and resealing of the at least one vessel occurs within the housing. In one or more embodiments, the caps are disposed within the housing. In one or more embodiments, the caps are at least partially disposed within the housing.

In one or more embodiments, resealing of the at least one vessel occurs within the housing. In one or more embodiments, the resealing occurs within a sterile compartment of the housing.

In yet a further aspect, the present invention provides a method of decontaminating a surface of at least one vessel, the method comprising: providing a decontamination device having a housing and a wiping member, the wiping member disposed within the housing and configured to move within the housing; providing at least one vessel having a surface; connecting the decontamination device to the at least one vessel; moving the wiping member within the housing, thus, decontaminating the surface of the at least one vessel; and detaching the at least one vessel from the device after the surface of the at least one vessel has been decontaminated.

In yet a further aspect, the present invention provides a method of decontaminating a surface of at least one vessel, the method comprising:
providing a decontamination device having a housing, a wiping member and one or more ports for receiving one or more vessels, wherein the wiping member disposed within the housing and configured to move within the housing;
providing at least one vessel having a surface;
connecting the decontamination device to the at least one vessel, by pushing the at least one vessel into the port of the decontamination device; and
moving the wiping member within the housing, thus, decontaminating the surface of the at least one vessel.

In one or more embodiments, the movement of the wiping member is exerted by the pushing motion of the at least one vessel into the port of the decontamination device.

In one or more embodiments, the device further comprises an actuator configured to move the wiping member across the housing following pushing the at least one vessel into the port of the decontamination device. In one or more embodiments, the actuator is selected from a group consisting of a lever, a gear, a spring, a mechanical actuator, an electrical actuator, a string, a clamp, a pin and combinations thereof.

In one or more embodiments, the device further comprises an actuator configured to move the wiping member across the housing at about the time of pushing the at least one vessel into the port of the housing. In one or more embodiments, the actuator is selected from a group consisting of a lever, a gear, a spring, a mechanical actuator, an electrical actuator, a string, a clamp, a pin and combinations thereof.

In one or more embodiments, the actuator is connected to the wiping member at one end thereof and having a second end or portion that protrudes into a lumen of the port such that when the vessel is pushed into the decontamination device, the second end or portion transfers the push forces to the actuator to thereby exert movement of the wiping member across the housing.

In one or more embodiments, the method further comprising engaging the at least one vessel to the decontamination device via an engagement mechanism. In one or more embodiments, the engagement mechanism is selected from a group consisting of a thread mechanism, a luer mechanism, a ratchet teeth/retention member mechanism, an adhesive mechanism, and combinations thereof.

In one or more embodiments, the method comprises resealing the surface of at least one vessel.

In one or more embodiments, resealing the at least one vessel from the device occurs prior to detaching the at least one vessel from the device.

In one or more embodiments, the method includes transferring fluid within the at least one vessel through the device after the surface of the at least one vessel has been decontaminated.

In one or more embodiments, the connection of the decontamination device to the at least one vessel results in a hermetic seal being formed between the decontamination device and the at least one vessel.

In one or more embodiments, the hermetically sealed connection between the housing and the at least one vessel is isolated from environmental/contaminated air.

In one or more embodiments, the at least one vessel is a syringe. In one or more embodiments, the at least one vessel is a container. In one or more embodiments the container is a bottle. In one or more embodiments, the container is a vial.

In one or more embodiments, the decontamination device further comprises a safety mechanism configured to prevent the detachment of the at least one vessel prior to resealing the surface of the at least one vessel. In accordance with this embodiment, the safety mechanism guarantees resealing of the at least one vessel prior to detachment from the decontamination device.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C are front cut views and FIG. 1D is a side cut view of illustrations presenting an exemplary system which includes a housing a wiping member disposed within the housing and two ports for allowing an engagement with two pharmaceutical vessels, the wiping member being moveable from an initial position and reaches an end position, thereby wiping off contaminants on either or both vessels, according to some embodiments of the invention.

FIG. 2 is a front cut view of an exemplary embodiment of the system which is configured for contacting and decontaminating various types of pharmaceutical vessels, according to some embodiments of the invention.

FIGS. 3A-3B are front cut views illustrations presenting an exemplary embodiment of the system which may further include a locking member configured to lock the wiping member at an end position, according to some embodiments of the invention.

FIGS. 4A-4B are front cut views illustrations of a further exemplary system which includes an opening in a wall of the housing, according to some embodiments of the invention.

FIG. 5 is a front cut view illustration presenting yet a further exemplary system which includes a filter disposed in an aperture in the housing, for releasing air from the housing when actuating the wiping member, according to some embodiments of the invention.

FIGS. 6A-6B are front cut view illustrations presenting yet a further exemplary system which includes an actuator in the form of a button which actuates the wiping member to slid across the housing by a pushing motion, according to some embodiments of the invention.

FIG. 7 is a front cut view illustration presenting yet a further exemplary system which includes a housing with a plurality of ports configured to connect to the housing a plurality of vessels, according to some embodiments of the invention.

FIG. 8 is a front cut view illustration presenting yet a further exemplary system which includes two housings disposed one above the other, according to some embodiments of the invention.

FIGS. 9A-9D are front cut view illustrations presenting yet a further exemplary system which includes two ports disposed on parallel longitudinal axes, according to some embodiments of the invention.

FIGS. 10A-10D are front cut view illustrations presenting yet a further exemplary system which includes a capping mechanism, according to some embodiments of the invention.

FIGS. 11A-11D are front views of yet a further exemplary system which includes an engagement mechanism in the form of an adhesive, for engaging a vessel to the housing, according to some embodiments of the invention.

FIGS. 12A-12B are front views of yet a further exemplary system which includes a port via which the vessel may be coupled to the housing, according to some embodiments of the invention.

FIGS. 13A-13B show the system of yet a further exemplary system which includes an engagement mechanism in the form of a thread and a complementary thread, for engaging a vessel to the housing, according to some embodiments of the invention.

FIGS. 14A-14D are front views of yet a further exemplary system which includes an engagement mechanism in the form of a clamping rail, for engaging a vessel to the housing, according to some embodiments of the invention;

FIGS. 15A-15B are front views of yet a further exemplary system which includes an engagement mechanism in the form of ratchet teeth for engaging a vessel to the housing, according to some embodiments of the invention.

FIGS. 16A-16F are front views of yet a further exemplary system which includes a moveable wiping member and a rail mechanism allowing an alignment of two vessels with respect to each other, according to some embodiments of the invention.

FIGS. 17A-17D are front views of yet a further exemplary system which includes an engagement mechanism in the form of a thread and a complementary thread, according to some embodiments of the invention.

FIGS. 18A-18F are front views of yet a further exemplary system which includes a sound alert in alternative states, according to some embodiments of the invention.

FIG. 19 is a perspective view of an exemplary system as herein disclosed, according to some embodiments of the invention.

FIGS. 20A-20C are front cut views of yet a further exemplary system which demonstrates an alternative embodiment of the capping mechanism, according to some embodiments of the invention.

FIGS. 21A-21D are front cut views of yet a further exemplary system which demonstrates an alternative embodiment of the wiping member, according to some embodiments of the invention.

FIGS. 22A-22D are front cut views of yet a further exemplary system demonstrating an alternative embodiment of the capping mechanism and a double wiper, according to some embodiments of the invention.

FIG. 23 demonstrates a side view of yet a further exemplary system, according to some embodiments of the invention.

FIGS. 24A-24C are front cut views of yet a further exemplary system demonstrating an alternative embodiment of exerting movement of a wiping member along a housing of a decontamination device, according to some embodiments of the invention.

FIGS. 25A-25C are front cut views of yet a further exemplary system demonstrating an alternative location of an actuator for exerting movement of the wiping member, according to some embodiments of the invention.

FIGS. 26A-26C are front cut views of yet a further exemplary system demonstrating another alternative location of an actuator for exerting movement of the wiping member, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary decontamination devices for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates to devices, methods and systems allowing an engagement of, and fluid passageway between vessels in a sterile or decontaminated manner. The present devices, methods and systems are particularly useful for medical purposes, wherein sterile preparations of medical substances are vastly needed. In one embodiment, the present invention provides systems configured to allow a substantially decontaminated engagement between vessels. In one embodiment, the present invention provides systems configured to decontaminate one or more surfaces of one or more vessels. In one or more embodiments, the systems of the invention allow a substantially decontaminated transferring of fluids between the one or more vessels. In one or more embodiments, the systems or devices allow the air-free or ambient air particle-free engagement between vessels

The invention provides a solution to an unmet and long felt need in medical settings and allows connecting in a sterile manner, two or more vessels. The herein disclosed devices and systems are user friendly, cost effective and abolish the need for complicated and expensive known methods for transferring medical substances.

In an aspect of the invention, the devices and systems of the invention include a housing having a side wall, a top wall and a bottom wall, and a wiping member disposed within the housing. In an embodiment of the invention, the top wall and/or bottom wall of the housing have at least one port or openings for receiving the vessels. The ports or openings may be formed by vertical walls extending from the top and/or bottom walls, forming an inner channel via which medical substances may be transferred following engagement between, and decontamination of the vessel surfaces. In one or more embodiments, the ports or openings have non-vertical walls. In one or more embodiments, the ports or openings may be flush mounted to the housing. In one or more embodiments, the ports or openings may be surface mounted to the housing. In one or more embodiments, the ports or openings may be concealed within at least a portion of the housing. In one or more embodiments, the ports or openings may be concealed within the housing.

In an embodiment of the invention, the wiping member is operable to move across and wipe off any contaminates present on the one or more surfaces of the vessels via an actuator. In one or more embodiments, the actuator extends through an opening of the housing. The actuator is configured to manipulate movement of the wiping member across the housing. In one or more embodiments, the wiping member is operable to move across and wipe off any contaminants present on the one or more surfaces of the vessels via an actuator extending through an opening in the side wall of the housing. In one or more embodiments, the actuator may extend through a top or bottom wall of the housing. In one or more embodiments, the wiping member is operable to move within the housing to wipe off any contaminants present on the one or more surfaces of the vessels.

In one or more embodiments, the actuator is operable to move the wiping member following pushing forces exerted by pushing a vessel into the housing (and/or into a port of the housing) of the decontamination device. In accordance with this embodiment, the push forces exerted by pushing a vessel into a decontamination device are transferred to the actuator which in turn moves the wiping member along the housing. Optionally, the actuator is operable automatically by pushing a vessel into the housing of the decontamination device. Alternatively, additionally or optionally, the actuator is operable manually by a user.

Various locations of the wiping member actuator are contemplated. For example, the actuator may be located within, outside, or partially within and partially outside the housing of the decontamination device.

Various types of actuators are contemplated for allowing manipulation of the wiping member to decontaminate a surface of a vessel. In one or more embodiments, the actuator is connected to the wiping member and extends externally via an opening within the housing. Exemplary actuators include, without limitation, a handle, a tab, a button, a touch button, a lever, a gear, a spring, a mechanical actuator, an electric actuator, an air pressure actuator, a string, a wire, a pin, a clamp, and combinations thereof. Optionally, the actuator is connected to the wiping member at one end thereof and extends into a port of the housing at another end or portion thereof. In one or more embodiments, the wiping member is operable to move across and wipe off any contaminates present on the one or more surfaces of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member. In one or more embodiments, the actuator is further configured to maintain the position of the wiping member in the initial position and allow the movement to the end position following the engagement of a vessel with the housing. In one or more embodiments, pushing a vessel into the housing bends or distorts the actuator such to release holding in the initial position and allow the movement to the end position. In one or more embodiments, the movement of the wiping member between the positions is reversible. In one or more embodiments, the movement of the wiping member between the positions is non-reversible and the wiping member is prevented from returning to the initial position. In one or more embodiments, a locking member may be disposed in or on the housing to prevent the wiping member to return to its initial position.

In an embodiment of the invention, the herein disclosed devices and systems are disposable. In an embodiment of the invention, the herein disclosed devices and systems are non-disposable.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria.

In one or more embodiments, the one or more of the surfaces of the one or more vessels is flat and/or smooth. The surface may be substantially flat or partially flat or sufficiently flat, allowing efficient decontamination thereof by the wiping member. In one or more embodiments, the one or more surfaces of the wiping member may be uneven or rugged.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof. In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings and/or ports of the decontamination device are sealed and/or covered thus preventing the sterilizing substance from escaping from inside the housing of the decontamination device. The seals and/or covers maintain the sterility of the decontamination device until about the time of use of the decontamination device.

In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

Various sizes and shapes of the housing are applicable. For example, the housing may have an external most length, width, and/or height of less than about 3 inches. In one or more embodiments, the housing may have an external most length, width, and/or height of less than about 25 millimeters (mm). For example, less than about 20 mm, less than about 15 mm, or less than about 10 mm. The housing internal most length, width, and/or height may be less than about 10 mm. For example, less than about 4 mm, less than about 3 mm, or less than about 2 mm. The housing may include one or more compartments which may optionally be separated or divided by the wiping member or by any other means. In one or more embodiments, the housing includes two compartments, wherein a first compartment is kept sterile and a second compartment is configured to receive a vessel which may be contaminated. In one or more embodiments, the sterile compartment can receive a vessel following decontamination of the surface of the vessel. In one or more embodiments, the housing includes two compartments wherein a first includes a capping mechanism and the second is configured to receive a vessel. In one or more embodiments, the housing includes three compartments wherein a first compartment is kept sterile, a second compartment is configured to receive at least one vessel which have a contaminated surface, and a third compartment having a capping or resealing mechanism. In one or more embodiments, the compartment having the capping or resealing mechanism is sterile. In one or more embodiments, the compartment having the capping or resealing mechanism contains a sterilizing or disinfecting substance. In one or more embodiments, the compartment configured to receive at least one vessel contains a sterilizing or disinfecting substance. In one or more embodiments, the compartment where the fluid passageway between vessels occurs contains a sterilizing or disinfecting substance. In one or more embodiments, the first compartment, the second compartment and/or the third compartment may have a pressure greater or less than an adjacent compartment. In one or more embodiments, the pressure may be greater or less than ambient air pressure. In one or more embodiments, the pressure may be greater or less than atmospheric pressure. In one or more embodiments, the pressure differential between compartments may be at least 0.02 i.w.c. (inches of water column). In one or more embodiments, the pressure differential between compartments is at least 0.1 i.w.c. In one or more embodiments, the pressure differential between compartment is between 0.01 i.w.c. and 1 i.w.c. In one or more embodiments, the pressure differential between compartments may be at least negative 0.05 i.w.c. In one or more embodiments, the pressure differential between compartments is between negative 0.4 i.w.c. and negative 0.01 i.w.c. As used herein, the term “i.w.c.” encompasses the definition as provided or accepted by the United States Pharmacopeia, The American Society of Health-System

Pharmacists, American Pharmacist's Association, and/or a nationally/internationally recognized organization or standardization organization.

In one or more embodiments, the housing is conveniently light presenting a weight of no more than about 100 grams (gr). For example, less than about 75 gr, less than about 50 mm gr, less than about 25 gr, less than about 10 gr, or less than about 5 gr.

In one or more embodiments, the housing may be transparent allowing visualization of its interior, or opaque.

In one or more embodiments, the housing may be a closed chamber which forms a tight, optionally, airtight connection between the vessels. In one or more embodiments, when the ports or openings are connected to vessels or when the ports are covered by a cover or a seal, the housing may form an interior which is hermetically sealed/isolated from surrounding/environmental air. In one or more embodiments, the cover or the seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing is already connected to one or more medical vessels. In accordance with this embodiment, the one or more medical vessels may be integrally manufactured or assembled with the housing and provided or distributed to consumers as such. In accordance with this embodiment, the system may be configured to allow a connection to additional one or more vessels. In one or more embodiments, the decontamination device abuts a wall of a container. In one or more embodiments, the decontamination device is flush mounted to a wall of a container. In one or more embodiments, the decontamination device is surface mounted to a wall of a container. In one or more embodiments, the container is a bag. In one or more embodiments, the container is a bottle. In one or more embodiments, the container is a vial. In one or more embodiments, the vessel is a syringe. Alternatively, the system of the invention is provided without any attachment to a vessel and is configured to connect a plurality of vessels and decontaminate thereof.

In one or more embodiments, the at least one vessel ranges from one vessel to ten vessels, wherein a surface of at least one or each of the vessels is configured to be wiped off by the wiping member.

In one or more embodiments, the system includes two or more housings each containing a wiping member. Optionally, the housings are disposed one above the other, providing a top housing and a bottom housing. In accordance with this embodiment, a wiping member disposed on the top housing is configured to wipe a surface of a vessel connected to the top housing and a wiping member disposed on the bottom housing is configured to wipe a surface of a vessel connected to the bottom housing.

In one or more embodiments, the system includes two or more ports which are disposed one above the other, or which are longitudinally aligned with respect to each other. In accordance with this embodiment, the vessels connected to such ports are positioned aligned allowing a direct and aligned fluid passageway between the vessels. In one or more embodiments, the system includes two or more ports which are shifted from each other. Yet, in accordance with this embodiment, a fluid passageway is formed between vessels which is twisted or curved.

In yet one or more embodiments, the housing contains a plurality of ports configured for connecting a plurality of vessels to the housing. The system or device is configured so that actuating the wiping member allows wiping the surfaces of all vessels connected to the housing in one action.

In one or more embodiments, the housing has an engagement mechanism configured or adapted to connect the at least one vessel to the housing. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In an embodiment of the invention, the engagement mechanism positions the surface of the at least one vessel at about same level as an interior wall of the housing. In an embodiment of the invention, the engagement mechanism positions the surface of the pharmaceutical vessel at the same level as an interior wall of a housing of the system. In an embodiment of the invention, the engagement mechanism positions the surface of the at least one vessel in a manner that protrudes into the housing. In an embodiment of the invention, the protruding of the surface of the at least one vessel is a slight/minor protruding. In an embodiment of the invention, the protruding of the surface of the at least one vessel ranges from less than one eighth of an inch to less than half an inch. In an embodiment of the invention, the engagement mechanism positions the surface of the at least one vessel in a manner that is below an inner surface of the housing. In an embodiment of the invention, the positioning of the surface of the at least one vessel ranges from less than one tenth of an inch to less than a half an inch from the inner surface of the housing. In an embodiment of the invention, the engagement mechanism positions the surface of the at least one vessel slightly below an inner wall/surface of the housing while allowing the wiping member to wipe off the entire surface of the at least one vessel. Optionally, or alternately, the engagement mechanism may be configured or adapted to connect at least one opening or port of the housing of the decontamination device to a connector. In one or more embodiments, the connector may be configured to couple/engage with an opening or port of the decontamination device. In one or more embodiments, the housing may have a plurality of openings/ports configured to couple/engage with a plurality of connectors. In one or more embodiments, the connectors may be further configured to couple/engage with a vessel. The connectors may have a conduit disposed within the connectors, wherein the conduit provides a fluidic passageway between at least one vessel and the decontamination device. In one or more embodiments, the herein disclosed invention allows transferring medical substances in a contaminant-free, or in a substantially contaminant-free manner.

In one or more embodiments, the herein disclosed invention affords an engagement of vessels in a contaminant-free, or in a substantially contaminant-free manner. In one or more embodiments, the herein disclosed invention affords an engagement of vessels in an air-free, or in a substantially air-free manner.

In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner.

In one or more embodiments, the herein disclosed invention, allows to substantially decrease the chances to introduce contaminants within a medical substance, when preparing or administering medical substances for administration to patients.

As used herein the term “substances” refers to various types of materials that should be kept sterile. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the term “pharmaceutical substance”

As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. In an embodiment of the invention, the vessel is utilized for containing or housing a medical substance. Various types of vessels are contemplated. The vessel may be selected, without limitation, from a vial, a bag, a chamber, a bottle, and the alike. In an embodiment of the invention, the term vessel further encompasses elements that can be used to connect between vessels. In accordance with this embodiment, the vessel may be selected, without limitation, from a connector, a connector having a plurality of openings, a syringe, an infusion line, a tubing, an infusion line, a spike, a syringe, a filter, a port and a manifold.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between two containers, between two bottles, between a syringe and a bag, between a syringe and a container, between a syringe and a vial, between a connector and a bag, between a container and a bag, between a vial and a bag, between a container and an infusion line or between a syringe and a connector.

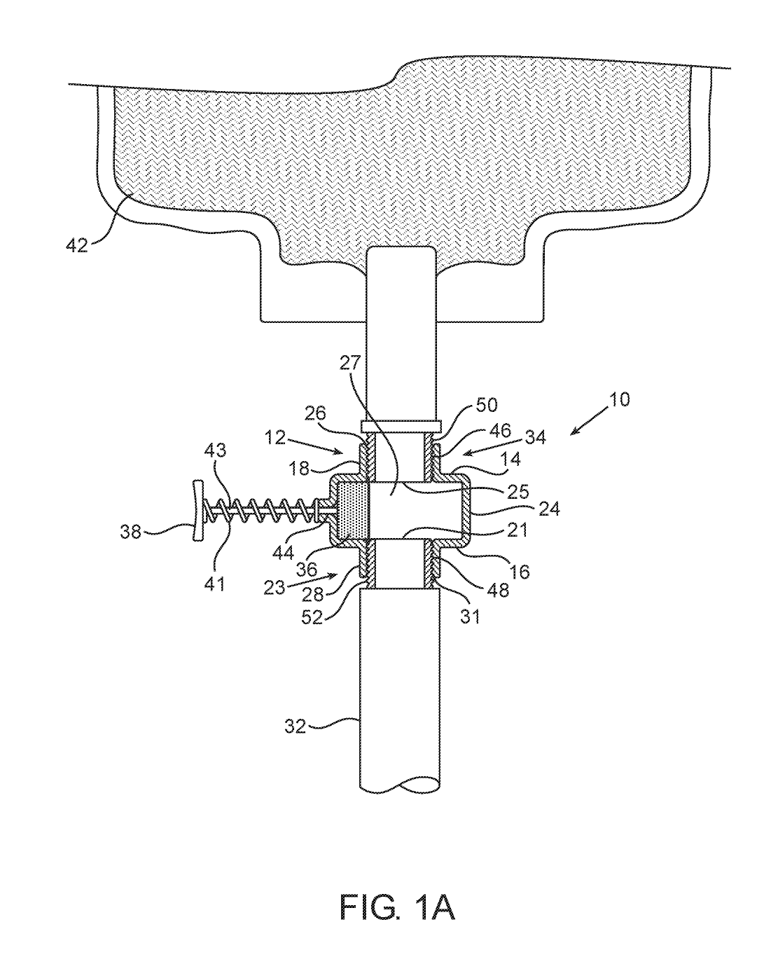

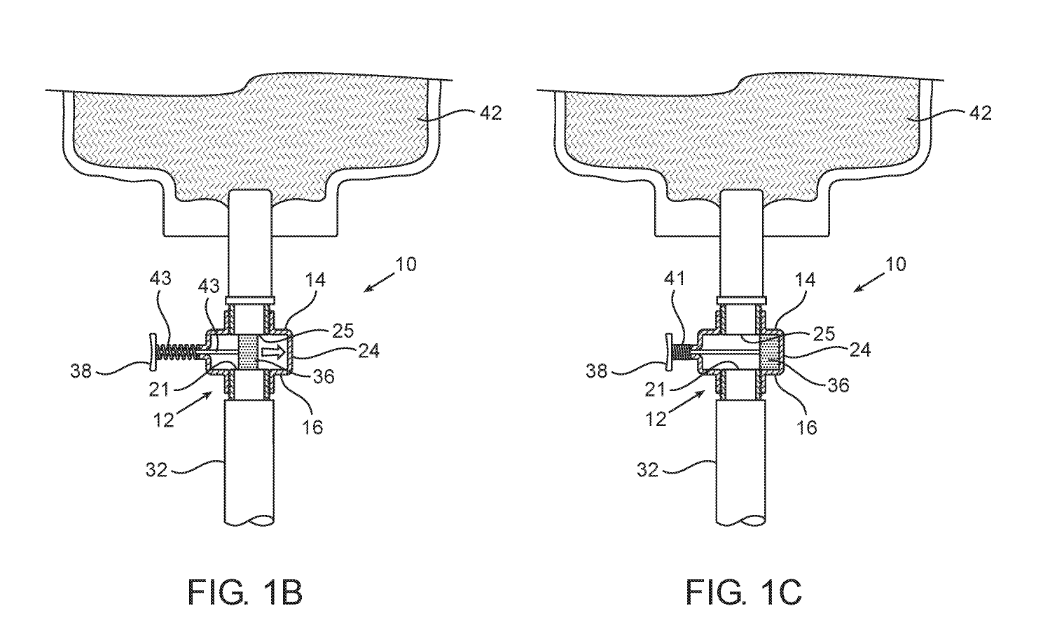

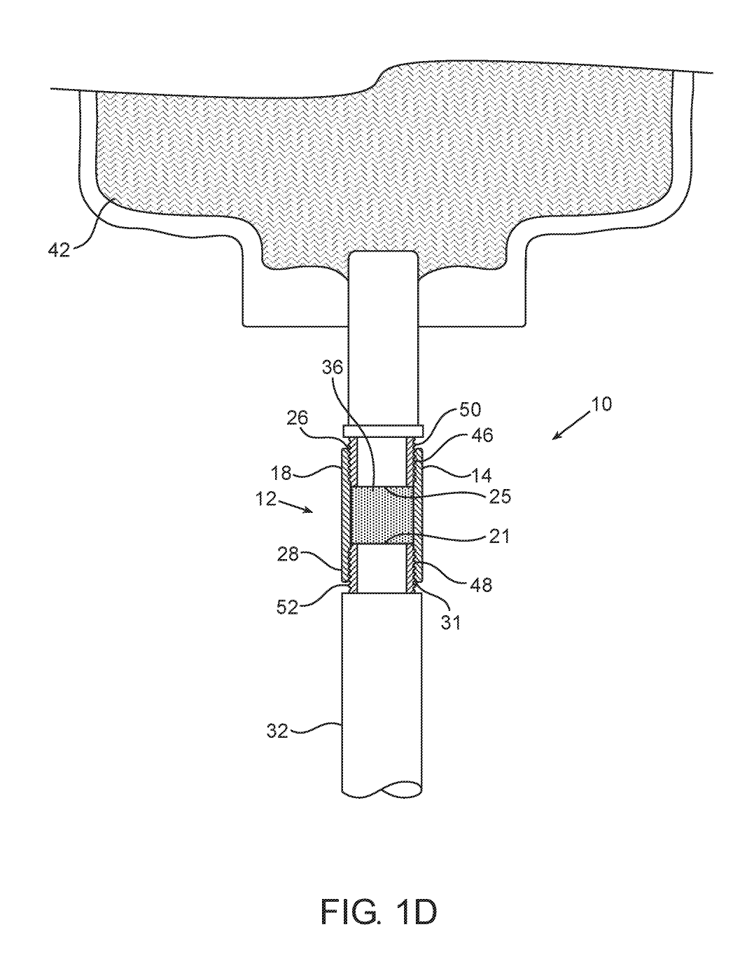

Referring now to the drawings, FIGS. 1A-1D illustrate system 10 for decontaminating medical vessels, such as syringe 32 and infusion bag 42. An infusion bag 42 and a syringe 32 are shown, but any type of pharmaceutical/medical vessel could be used and is herein contemplated, such as a bottle, a vial, an infusion line, a connector, a filter, a manifold, etc. System 10 includes a housing 12 having a side wall 24, a top wall 14 and a bottom wall 16. The housing 12 defines an inner lumen 27 via which fluid passageway can be formed between bag vessel 42 and syringe 32. The housing 12 further comprises a top port 34 with vertical side walls 18 extending upwardly from top wall 14 and defining an opening 26 of housing 12. Housing 12 further comprises a bottom port 23 with vertical side walls 28 extending downwardly from bottom wall 16 and defining an opening 31 of housing 12.

Top port 34 is configured to allow coupling to vessel 42 and bottom port 23 is configured to allow coupling to vessel 32. Optionally, system 10 is provided when housing 12 is connected already to, optionally integrally manufactured with, vessel 32 or vessel 42. Port 23 may be covered by a cover (not shown) providing a sealed system 10 prior to an engagement with vessel 32. Alternatively, port 34 may be covered by a cover (not shown) providing a sealed system 10 prior to an engagement with vessel 42. Openings 26 and 31 are sized and shaped to allow connection to vessels 42 and 32, respectively, in a tight, optionally, in an airtight manner.

Vertical side wall 18 includes internal threads 46, being configured for cooperating with external threads 50 on vessel 42, to thereby connect vessel 42 to housing 12. Alternately, vertical side wall 18 may include external threads (not shown) being configured for cooperating with internal threads (not shown) of vessel 42.

Additionally, or alternatively, vertical side wall 28 has internal threads 48, being configured for cooperating with external threads 52 on vessel 32, to connect the vessel 32 to housing 12. Optionally, or alternatively, vertical side wall 28 may have external threads (not shown) being configured for cooperating with internal threads (not shown) on vessel 32. Other engagement mechanisms for allowing an engagement with vessels may optionally include an adhesive (not shown) for allowing an adhesive engagement of one or both vessels 32 and 42 or ports thereof to the adhesive walls of ports 23 and 34, respectively, or to top wall 14 and bottom wall 16.

Disposed within housing 12 is a wiping member 36 configured to move across the housing 12 and to thereby decontaminate surfaces 25 and 21 of vessels 42 and 32, respectively. Wiping member 36 extends between internal top wall 14 and internal bottom wall 16 of housing 12. Wiping member 36 is manipulated from the exterior to move across the housing 12 by a wiping member actuator having a form of an actuator/handle 38. A transfer mechanism in the form of a rod 43 loaded by a spring 41 is configured to transfer a force from the actuator 38 to the wiping member 36 to move the wiping member 36 within the housing 12.

Wiping member 36 is connected to actuator/handle 38 via rod 43 which extends through an opening 44 in side wall of the housing 12. Rod 43 extends from opening 44 such that an airtight seal is maintained between rod 43 and the opening 44 in the side wall. Opening 44 may be lined internally or circumferentially with an elastomeric material that provides for an airtight seal between rod 43 and opening 44. The elastomeric material may be a plastic, a rubber, or another material.

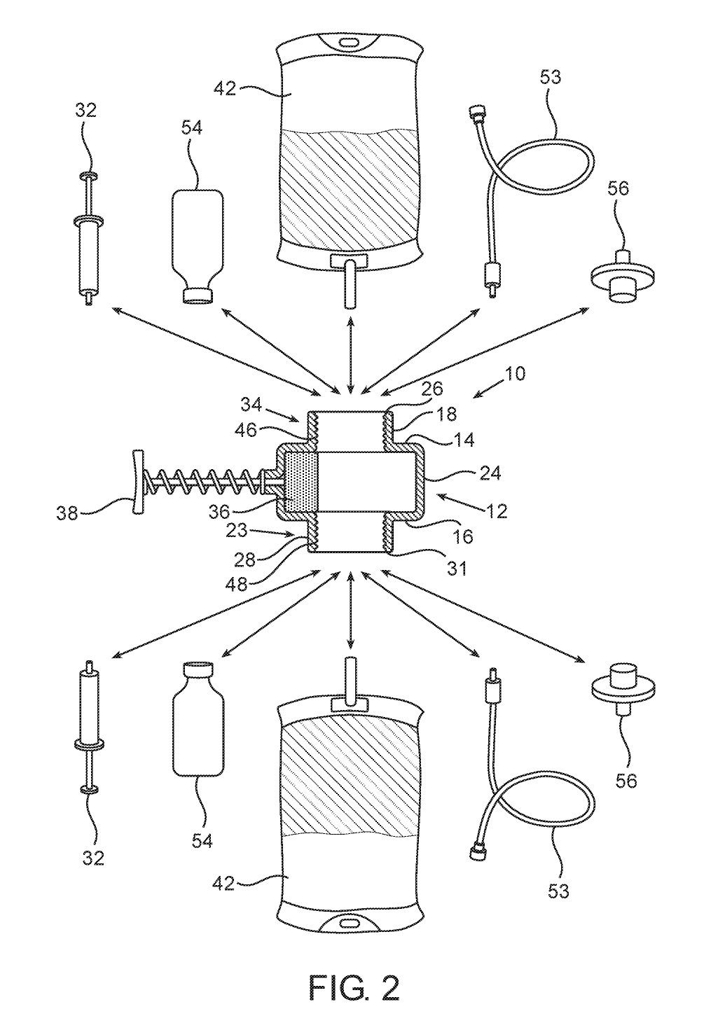

Actuator 38 is configured to allow movement of the wiping member 36 via a pushing and/or sliding motion by a user. The spring 41 loaded rod 43 tensions wiping member 36 in an initial position (shown in FIG. 1A) with wiping member 36 disposed at a first side end of housing 12. The action of wiping member 36 is shown in FIGS. 1B and 1C. Pushing actuator 38 forces wiping member 36 to partially or entirely rub across or wipe off the top surface 21 and/or 25 of syringe 32 and/or bag 42, respectively. Such pushing action moves wiping member 36 from the initial position (FIG. 1A) to an end position (FIG. 1C), wherein wiping member 36 disposed at a second opposing side end of housing 12 and wipes away any contamination of a dirt and/or pathogen such as bacteria that may have accumulated on top surface 21 and/or 25. FIG. 1D shows a cross sectional side view of system 10. Wiping member may expand to cover the entire surface 21 and/or 25 of vessels 32 and/or 42, forming a hermetic seal between wiping member 36 and surfaces 21 and/or 25.

[00141]Reference is now made to FIG. 2 which illustrates the herein disclosed system 10 that may allow an engagement and decontaminated fluid passageway between two vessels. The system 10 includes a top port 34 and a bottom port 23 each can be engaged with various types of vessels, such as syringe 32, bottle 54, infusion bag 42, infusion line, 53 and filter 56. Optionally, one or both ports 23 and 34 is attached to or integrally attached in a permanent or fixedly manner to the vessel(s). One of the ports 23 and 34 may be already attached, or coupled, in a decontaminated manner, to a vessel at about the time of manufacture and/or distribution of the system. One of the ports 23 and 34 may form a unitary structure with a vessel. Alternatively, both ports 23 and 34 may be manufactured or distributed without any attachment to a vessel. Openings 26 and 31 are sized and shaped to allow a tight connection, optionally an airtight connection, between ports 34 and 23, respectively, and any of the herein disclosed vessels.

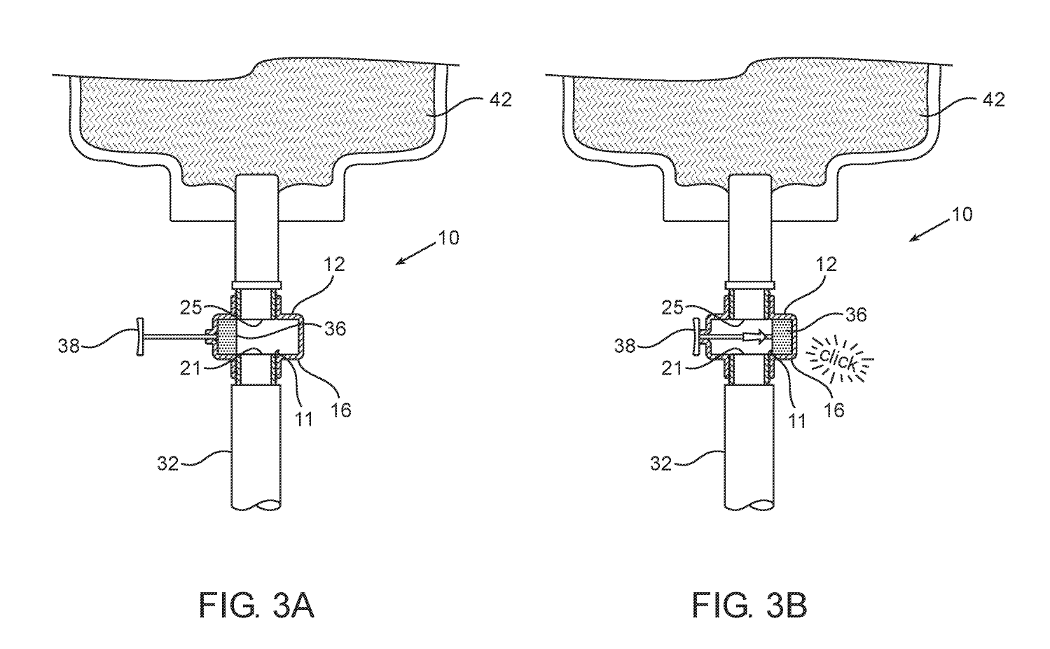

Reference is now made to FIGS. 3A-3B illustrating system 10 which further includes a locking member 11 disposed in the housing 12. The locking member 11 being configured for locking the wiping member 36 in the end position, following actuation of wiping, to prevent the wiping member 36 from returning to an original or an initial position. Locking member 11 is disposed on the internal surface of bottom wall 16. When wiping member 36 passes across surfaces 21 and 25, it passes over locking member 11, which is angled away from wiping member 36 and allows it to pass therethrough. Once wiping member 36 reaches the end position, shown in FIG. 3B, locking member 11 presses against wiping member 36, preventing it from returning backwards. Optionally, a click sound is heard when wiping member reaches the end position and upon locking thereof by locking member 11.

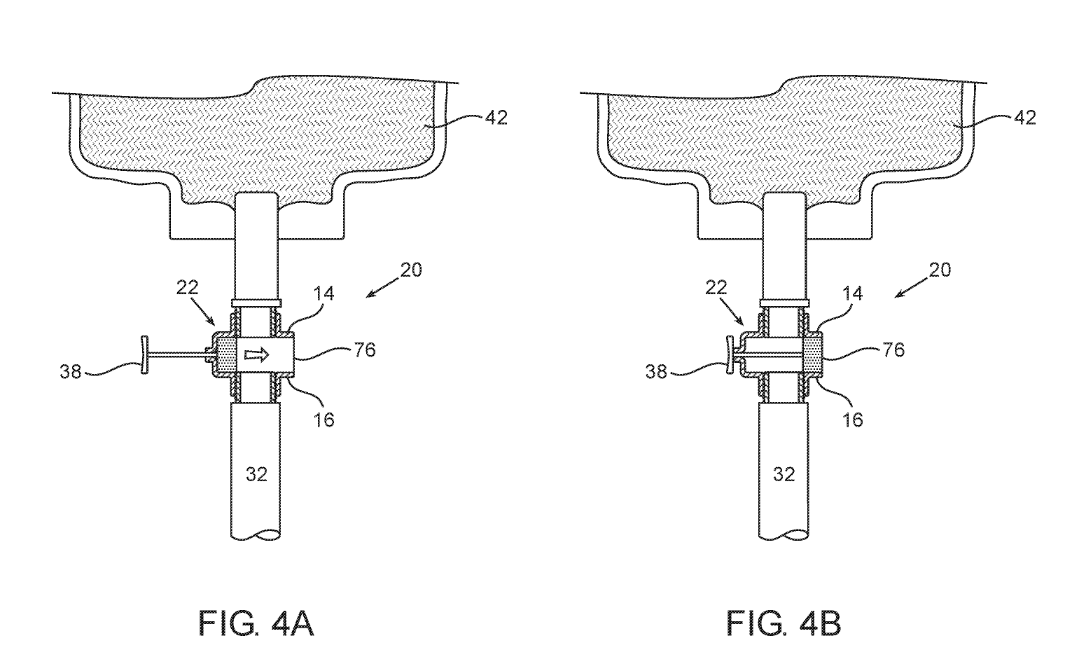

Reference is now made to FIGS. 4A-4B which illustrate system 20. System 20 is similar to system 10 but includes a housing 22 that is open on one side via opening 76. Pushing wiping member 36 across the housing 22, from a first side end (e.g., left) to a second side end (e.g., the right side), seals opening 76 by virtue of blocking it by wiping member 36. Wiping member 36 thus allows a hermetically sealed system 20.

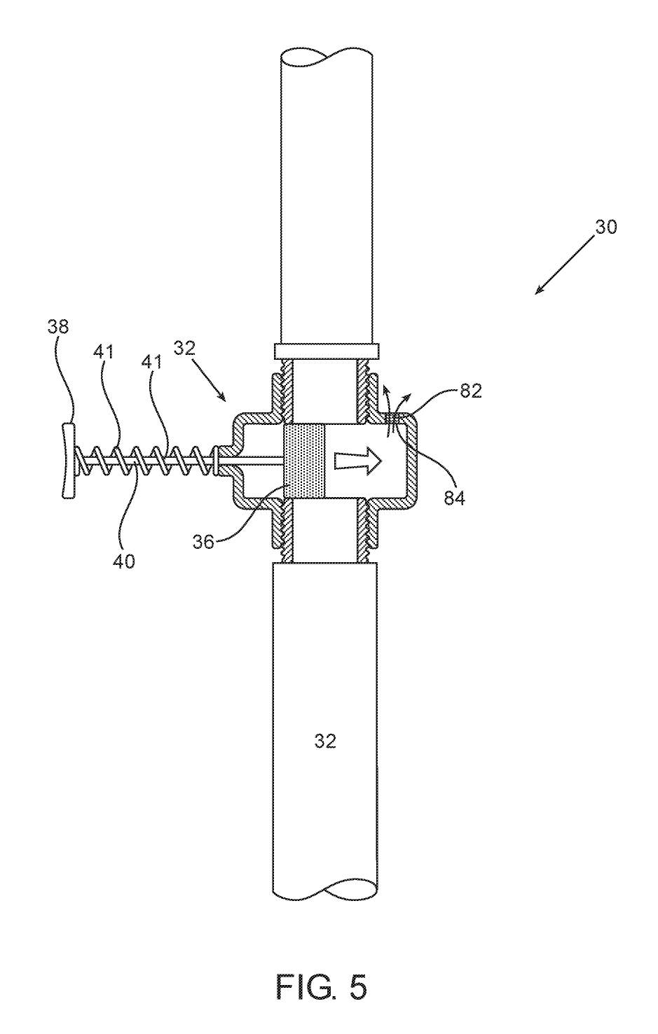

Reference is now made to FIG. 5 which illustrates system 30. System 30 is similar to system 10 but contains a housing 32 having a filter 82 disposed in an aperture 84 in the housing 32. Filter 82 is configured to allow for releasing air from the housing 32 when actuating the wiping member 36. Accordingly, air pressure that can be built up during movement of wiping member 36 can be prevented. Filter 82 is located and devised so as to allow the pressurized air to pass there through, without allowing any contaminants inside. Filter 82 may have pores having a diameter of less than about a few microns and can push contaminants out of the housing 12. Filter 82 may have a porosity of about 0.22 microns or less. Optionally, filter 82 may be a valve. The valve 82 may be a one-way valve allowing air to pass out of housing 32 while preventing ambient or environmental air from entering housing 32.

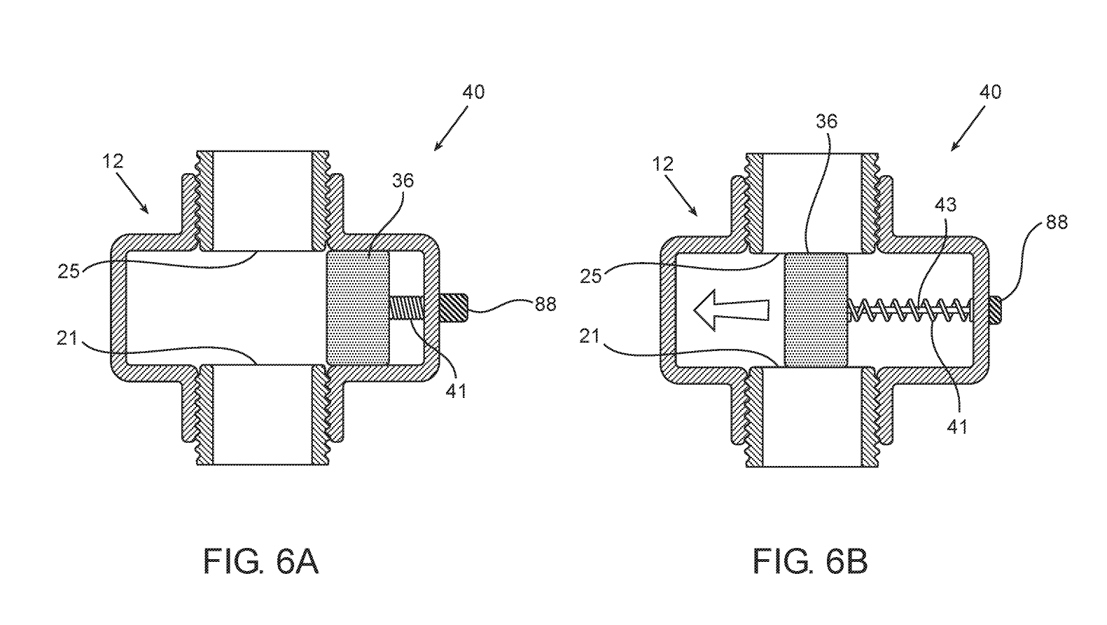

Referring now to FIGS. 6A-6B, system 40 is shown which is similar to system 10 but includes an actuator presenting the form of a button 88 attached to rod 43 surrounded by spring 41. The actuator 88 is actuated to manipulate sliding of wiper member 36 across housing 12 and wipe surfaces 21 and and/or 25 by pushing thereof.

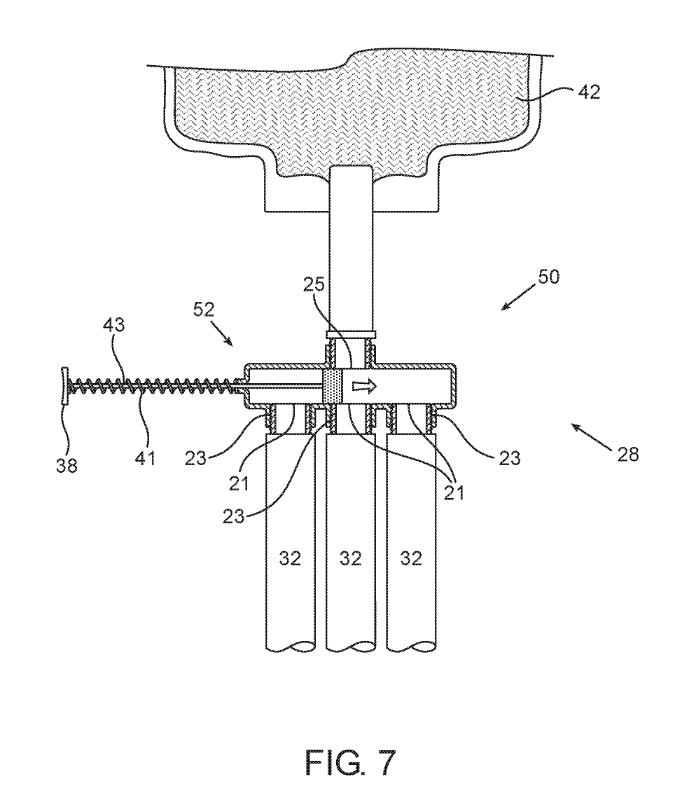

Reference is now made to FIG. 7 which illustrates a further exemplary system 50 wherein a housing 52 is made wider than housing 12 shown in FIGS. 1-3 and 6 and contains three bottom ports 23 arranged along bottom wall 16. This way, three vessels 32 can be connected to housing 52 and can have their surfaces 21 partially or entirely wiped off at about the same time, sequentially, or at the same time, in one action, by wiping member 36, when actuated by actuator 38 connected to rod 43 surrounded by spring 41. Optionally, each of the plurality of ports 23 provides airtight engagement between the housing 52 and vessels 32.

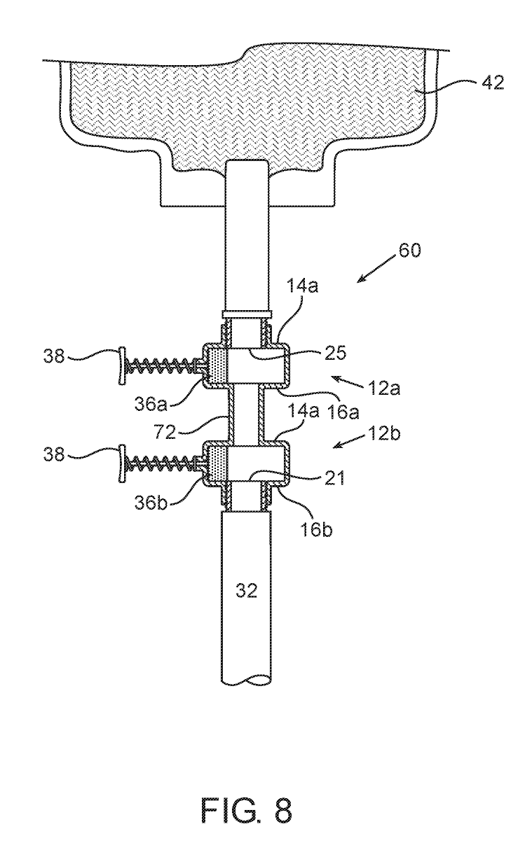

Reference is now made to FIG. 8 which illustrates a further exemplary system 60 that contains two housings 12, being housing 12a and housing 12b and two wiping members 36a and 36b disposed one above the other, wherein wiping member 36a is arranged to wipe surface 25 of vessel 42 connected to top wall 14a of the housing 12a, and wiping member 36b is arranged to wipe surface 21 of vessel 32 connected to bottom wall 16b of the housing 12b. Intermediate portion 72 connects the two housings 12a and 12b and is configured to allow fluid passage therethrough.

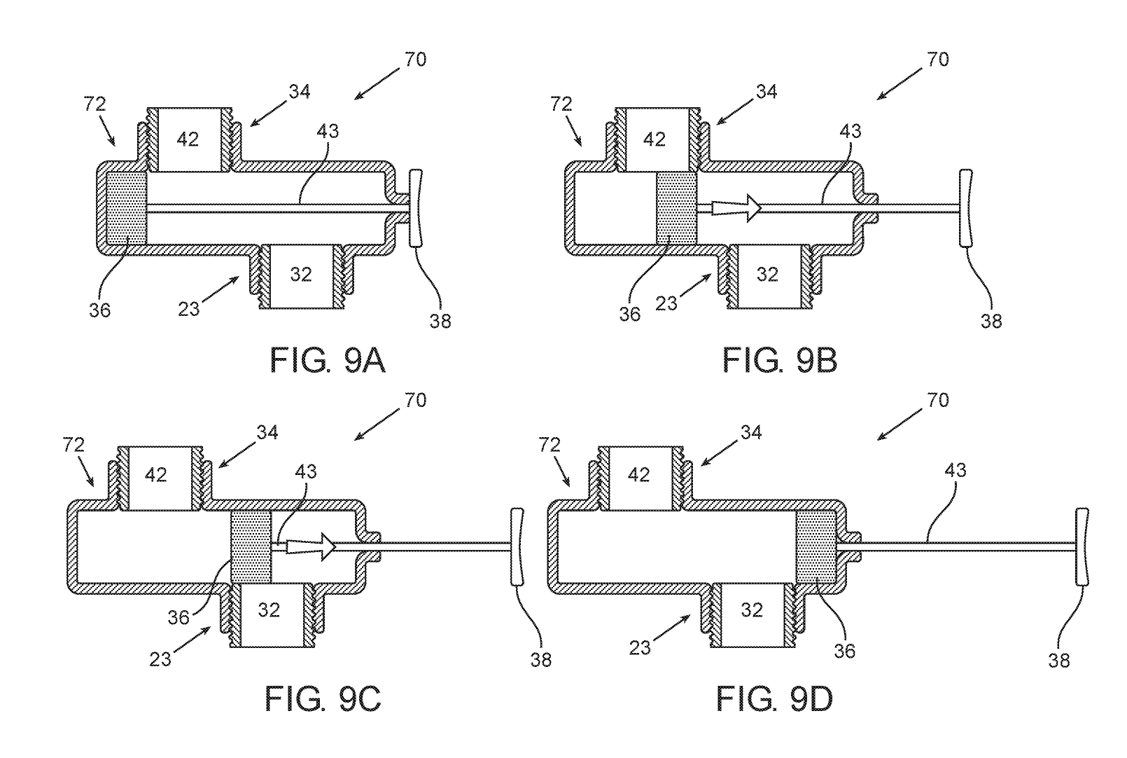

Referring to FIGS. 9A-9D, a further exemplary system 70 for decontaminating vessels is shown. System 70 includes housing 72 with ports 23 and 34. In this case ports 23 and 34 are shifted from each other and lie in parallel longitudinal axes. Each of ports 23 and 34 are configured to connect vessels 32 and 42, respectively. The system 70 further includes an actuator 38 being a handle connected to rod 43, but this time without a spring. The actuator 38 is operable to allow movement across the housing 72 by an axial pulling motion. Ports 23 and 34 are disposed in housing 72 such that they are shifted from each other. Still, a fluid passageway between the vessels 32 and 42 can be formed upon engagement thereof to system 70.

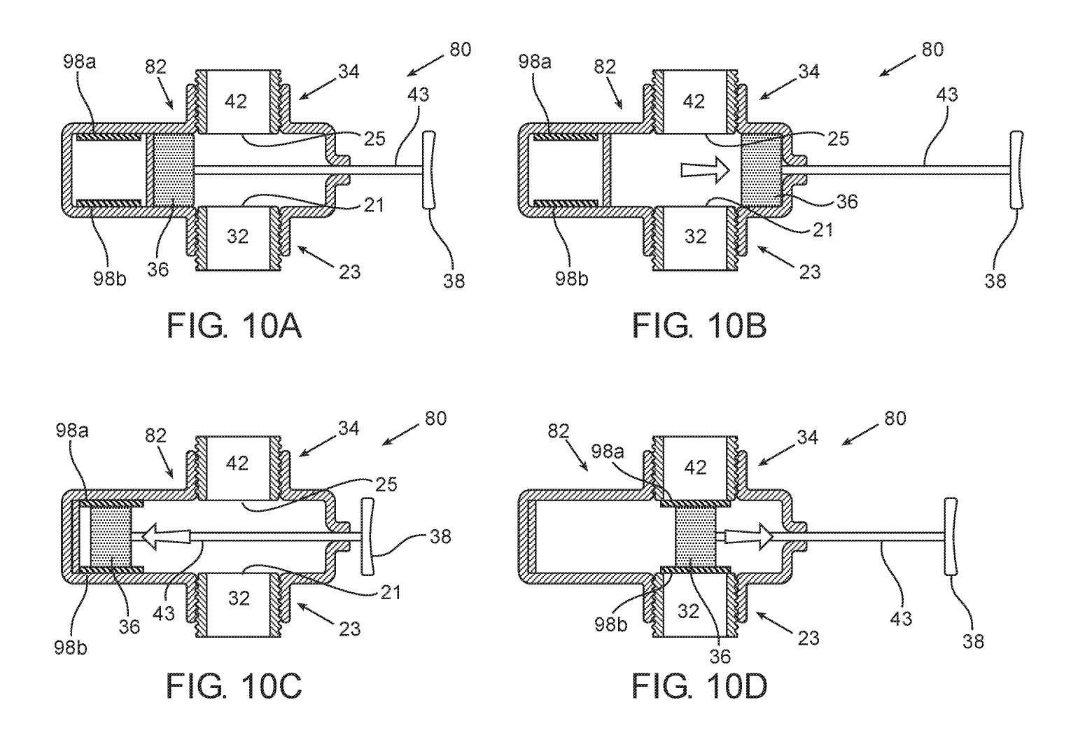

FIGS. 10A-10D present yet a further exemplary system 80 which includes a housing 82 with ports 23 and 34 and a capping mechanism 98 including top cap 98a and bottom cap 98b. The system 80 further comprises an actuator comprising a handle 38 attached to a rod 43. The actuator is operable to allow movement across the housing 82 by axial pulling/pushing motion of the handle 38 by a user. FIG. 10A shows wiping member 36 when disposed in an initial position. FIG. 10B shows wiping member 36 following decontamination of surfaces 21 and 25. The wiping member 36 is disposed in an end position. FIG. 10C shows wiping member 36 at a third position when moved to a resealing compartment within housing 82. Resealing compartment may be disposed on either of first side or second side ends of housing 82. Resealing compartment includes one or more top caps 98a and 98b. Wiping member 36 can attach to one or more of top caps 98a and 98b and slide there along housing 82 when attached thereto. In FIG. 10D wiping member 36 reseals or recaps vessels 42 and 32 with top caps 98a and 98b, respectively. Caps 98a and 98b are disposed within the housing and resealing of vessels 42 and 32 occurs within the housing 82.

FIGS. 11A-11D illustrate yet a further exemplary system 100 that includes a housing 112 with side walls 124 extending between bottom wall 116 and top wall 114. The housing 112 defines an inner lumen (not shown) via which fluid passageway is formed following engagement between bag 42 and bottle 54. The inner lumen includes a wiping member which can be similar to wiping member 36 shown in systems 10, 20, 30, 40, 60 and 80.

The wiping member 36 is actuated to wipe at least a portion or the entire surface of vessel 42 and 54. The actuation in this case is carried out via a wiping member actuator 138 having a tab-like structure. In system 100, vessel 42 may be integrally connected or form a unitary structure with housing 112 and provided as such. Optionally, housing 112 may abut a wall of vessel 42, may be flush mounted to vessel 42, may be surface mounted to vessel 42 or may share a wall with vessel 42. Optionally, vessel 42 may be a bag, a bottle, a syringe, an infusion line, a vial or a connector. FIG. 11A shows vessels 42 and 54 when spaced apart and vessel 42 is already provided connected to housing 112. Ambient air particles AA are present between vessel 54 and system 100. In FIG. 11B, the vessels 42 and 54 are connected via an engagement mechanism 115 being an adhesive. An adhesive 115a is attached at bottom wall 116 of housing 112. A second adhesive 115b may be present and attached to surface 121 of vessel 54. Although system 100 is illustrated herein when including two adhesives, the herein disclosed systems may alternatively include a single adhesive provided on either housing 112 or on vessel 54. Adhesives 115a and/or 115b may be peripheral, having a ring-like structure, allowing peripheral adhesion and direct contact between surface 121 and an opening (shown for example, in FIGS. 1A-1C) at the bottom wall 116 of system 100. The ring-like structure of adhesives 115a and/or 115b may be circumferential to an opening and/or port of vessel 54 and or housing 112. In FIG. 11C the actuator 138 moves the wiping member (not shown) which is disposed within housing 112 across the lumen of housing 112 and wipes off any contaminants present on surface 121. FIG. 11D shows a fluid passageway formed between vessels 42 and 54 upon actuating piercing member 97 to slide downwardly and pierce surface 121 of vessel 54, via piercing member actuator 101.

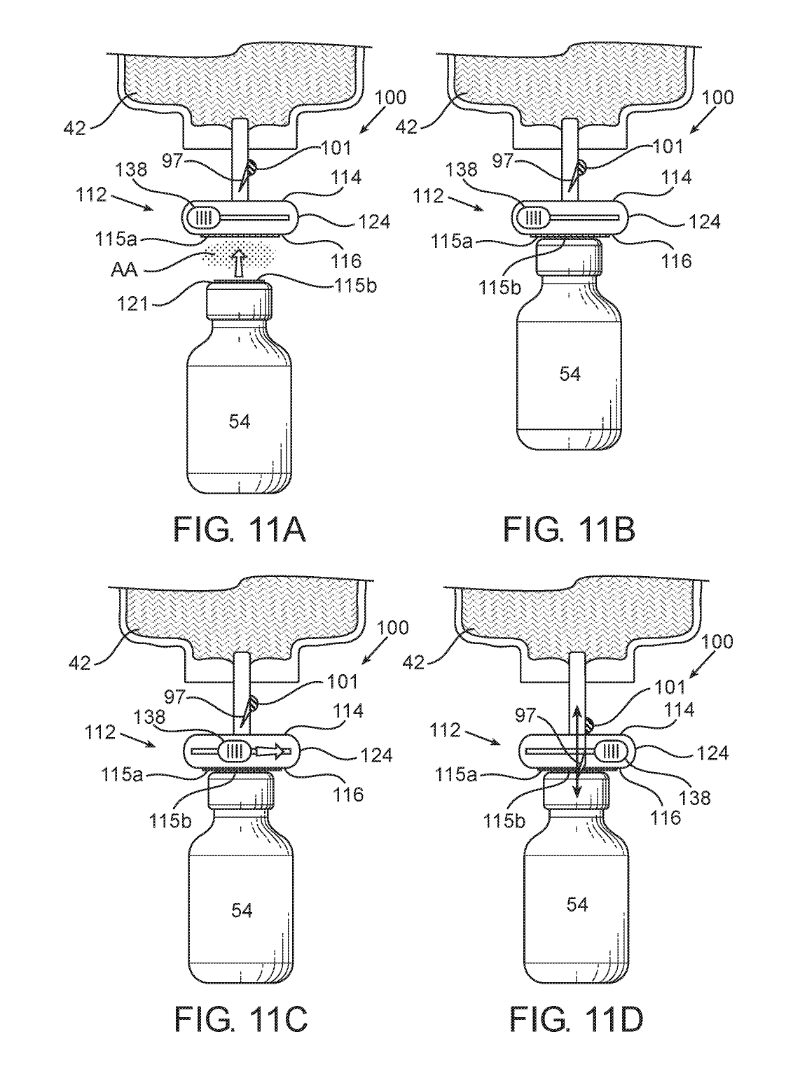

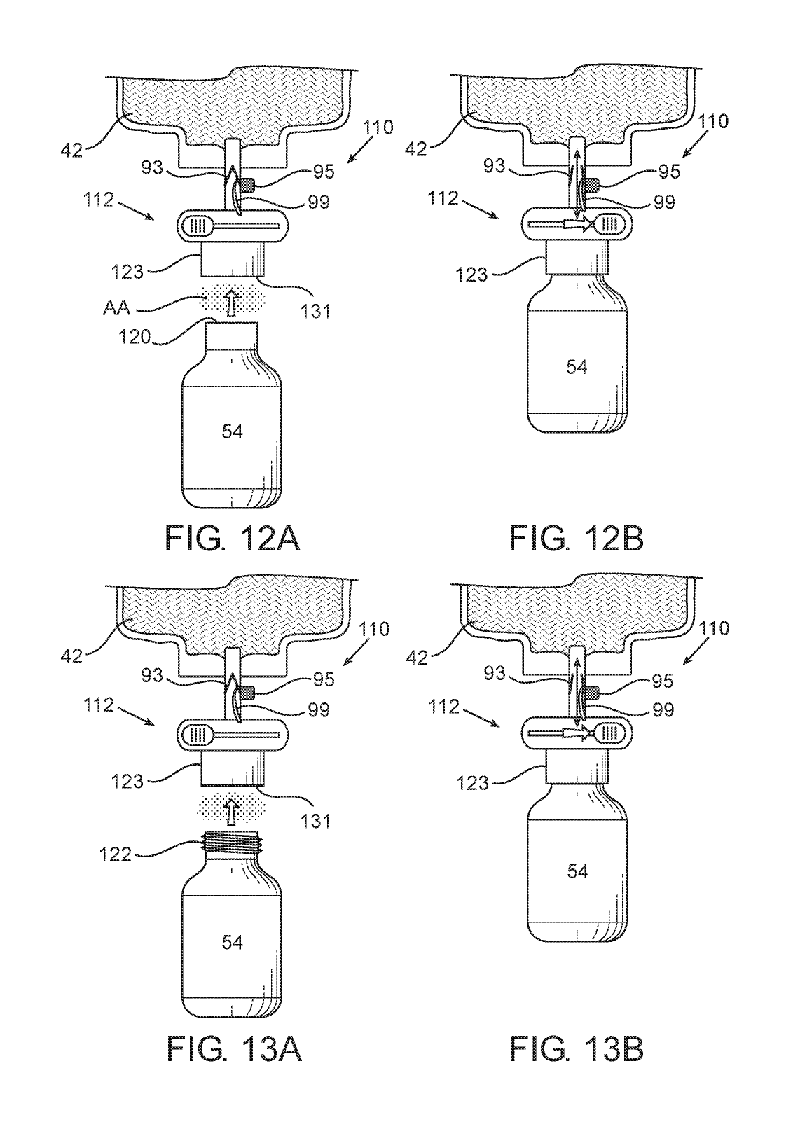

FIGS. 12A-12B present yet a further exemplary system 110 which is similar to system 100 but includes a bottom port 123 defining an opening 131 of housing 112. Bottom port 123 is configured to allow coupling of vessel 54 to housing 112. AA surrounding the surface 120 of vessel 54 is wiped off by a wiping member positioned in the interior of housing 112. System 110 further includes a valve 93, optionally a one way, or a two-way valve, that is closed prior to any decontamination action made by wiping member. Valve 93 may be operable also via valve actuator 95 which actuates valve unlocking mechanism 99. Optionally, valve actuator 95 may directly open valve 93. Valve 93 may be opened by the action of moving the wiping member disposed within the housing 112. Optionally, valve 93 may be opened by the action of moving the wiping member across the housing 112. Valve 93 is shown closed in FIG. 12A and opened in FIG. 12B. A fluid passageway is formed between vessel 42 and 54 following the movement and decontamination action of wiping member. In

FIGS. 13A-13B shows system 110 further comprising an engagement mechanism allowing engagement between vessel 54 and system 110 via a thread 122 disposed on vessel 54 that is complementary to a thread surrounding the internal of port 123 of system 110 (not shown).

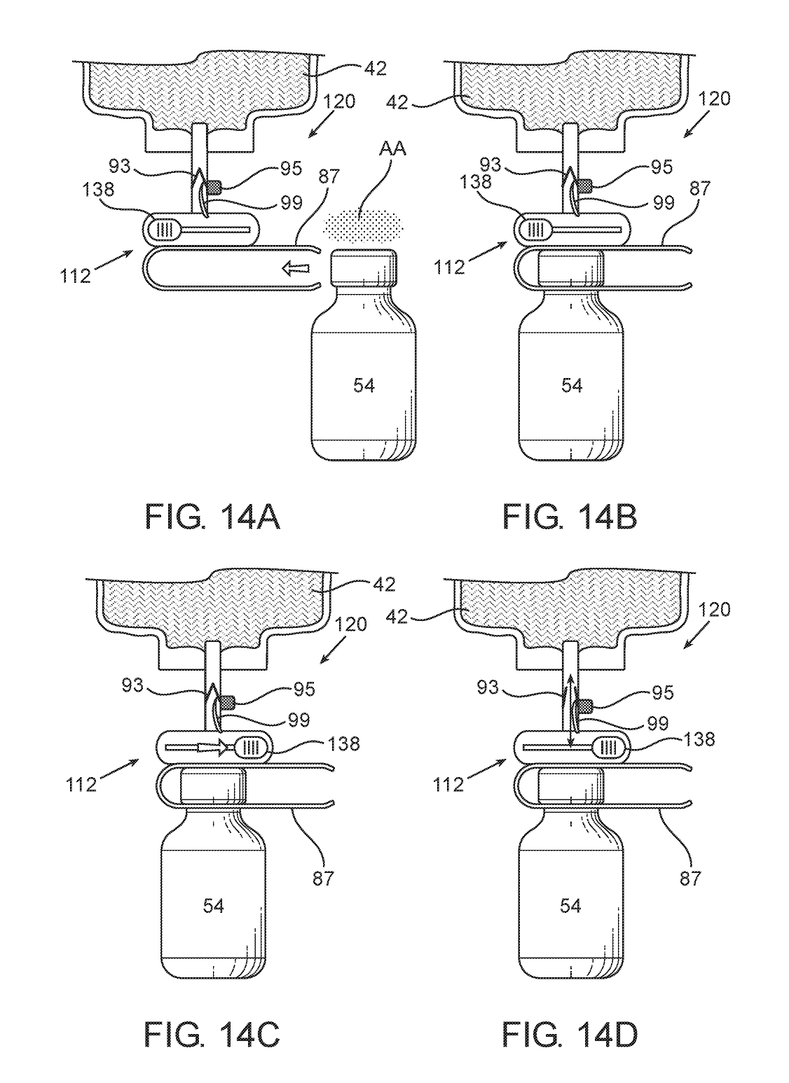

FIGS. 14A-14D present yet a further exemplary system 120 which is similar to systems 100 and 110 but includes an engagement mechanism allowing engagement between vessel 54 and system 120 via clamping rail 87. A port of vessel 54 can be engaged within clamping rail 87 and slide there along until reaching a longitudinal alignment with vessel 42. A fluid passageway can be formed between the vessels following opening of valve 93 by valve unlocking mechanism 99 and valve actuator 95. FIG. 14A shows vessel 54 and system 120 when spaced apart and AA surround the vessel 54. In FIG. 14B, vessel 54 slides along the clamping rail 87 mechanism until reaching a longitudinal alignment with vessel 42. The wiping member is then actuated by a user using wiping member actuator in the form of tab 138 (FIG. 14C). A fluid passageway can be formed upon opening of valve 93, optionally by an action mediated by a user that operates unlocking mechanism 99 to open the valve via valve actuator 95 (FIG. 14D). In an alternative embodiment, the valve 93 is opened at the time of movement of wiping member within housing 112. In accordance with this embodiment, the valve unlocking mechanism 99 can be operated to unlock the valve 93 by the movement across the housing of the wiping member. Optionally, the valve 93 may be opened by moving tab 138 across the exterior of housing 112.

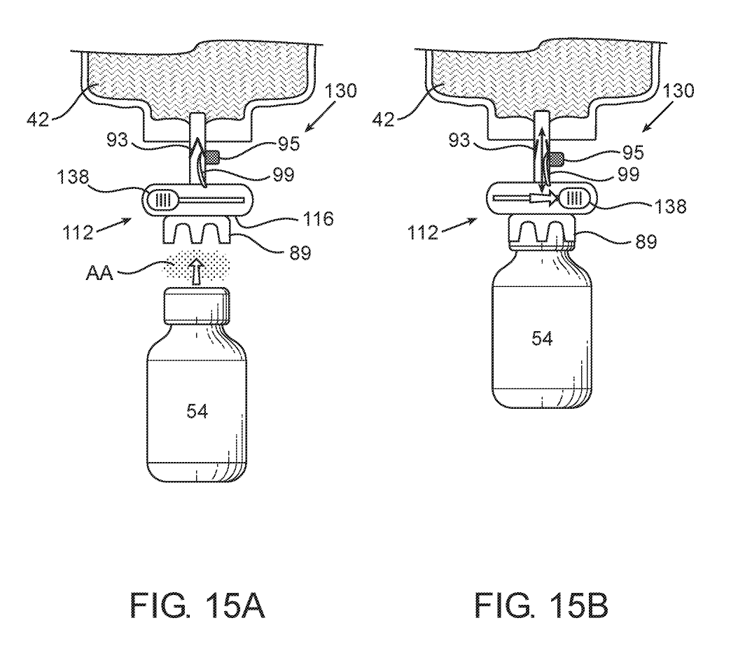

FIGS. 15A-15D demonstrate yet a further exemplary system 130 which is similar to system 120 but includes an engagement mechanism in the form of ratchet teeth 89. Ratchet teeth 89 are shown when disposed at the bottom wall 116 of system 130, but complementary ratchet teeth may be present on top of vessel 54.

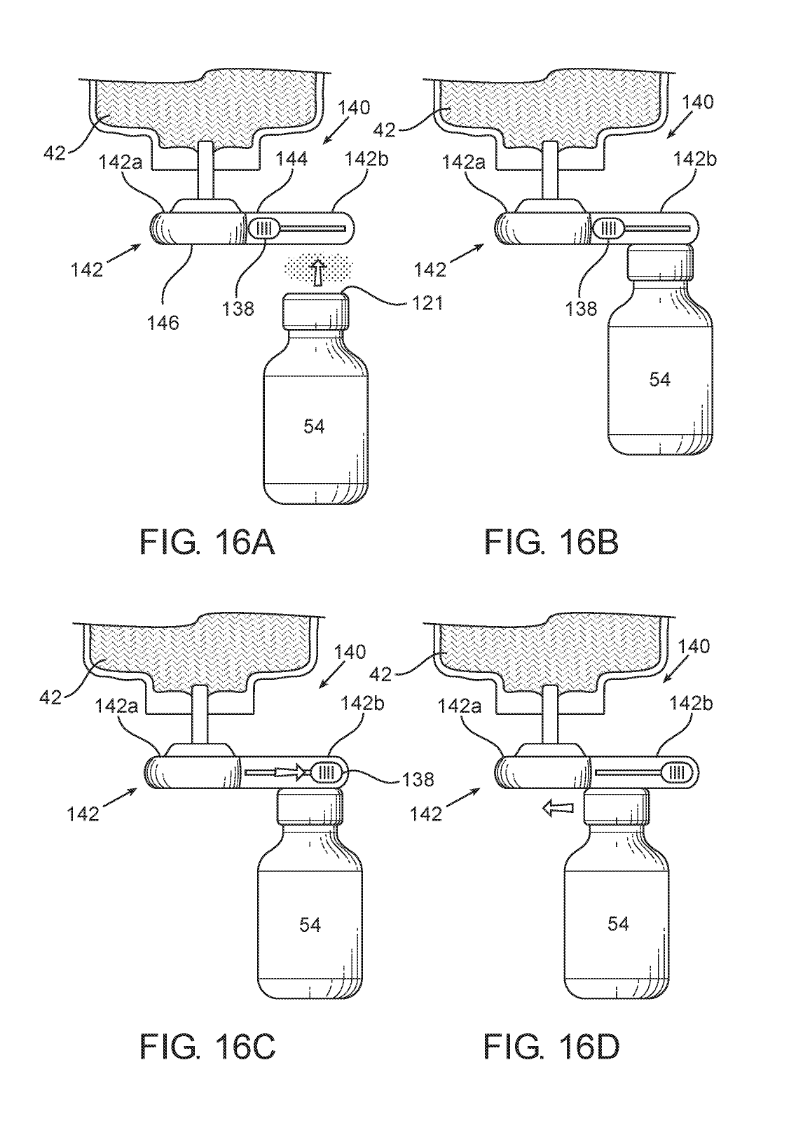

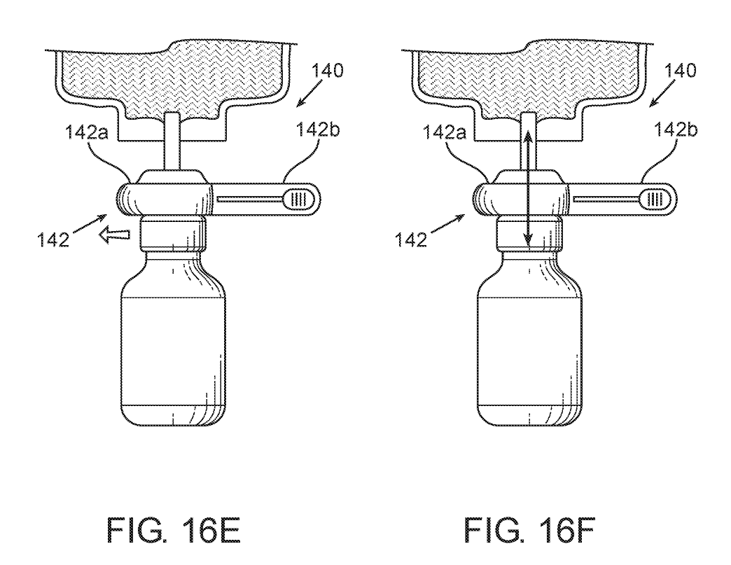

FIGS. 16A-16F demonstrate yet a further exemplary system 140 which includes a housing 142 with a first compartment 142a being decontaminated or sterile and a second compartment 142b allowing an engagement with vessel 54. First compartment 142a or second compartment 142b may contain or house a sterilizing or disinfecting substance. The sterilizing or disinfecting substance may be in liquid, gas, aerosolized, semisolid or solid form. Optionally, first compartment 142a may have a pressure greater or less than second compartment 142b. Alternately, second compartment 142b may have a pressure greater or less than first compartment 142a. Optionally, first compartment 142a may have a pressure greater or less than ambient air or atmospheric pressure. Optionally, second compartment 142b may have a pressure greater or less than ambient air or atmospheric pressure. A wiping member (not shown) is disposed between the compartments and/or within compartment 142b. Alternately, the wiping member (not shown) may be disposed within compartment 142a. The wiping member may be expandable extending between top wall 144 and bottom wall 146 of housing 142, allowing a hermetically sealed compartments 142a and/or 142b. An engagement between the housing 142 and vessel 54 and movement of vessel 54 is formed via a rail mechanism (not shown) allowing sliding there along of the vessel 54 until reaching a longitudinal alignment with vessel 42. FIG. 16A shows the vessels 54 and 42 when spaced apart and AA is present between the vessels. FIG. 16B shows an engagement between vessel 54 and housing 142 of system 140. FIG. 16C shows tab 138 while moved to allow wiping surface 121 of vessel 54 by a wiping member disposed within the housing. FIG. 16D shows the vessel 54 that slides via the rail mechanism (not shown) towards compartment 142a. FIG. 16E shows an alignment formed between vessel 42 and 54. FIG. 16F shows a fluid passageway formed between vessels 42 and 54. Fluid passageway formed between vessels 42 and 54 may occur via piercing of a surface of vessel 54 and/or breaking/rupturing of a frangible seal of vessel 54. The piercing of vessel 54 surface may occur via a piercing member (not shown) disposed within the housing 142 of system 140 and/or via a piercing member (not shown) disposed within vessel 42 and/or 54.

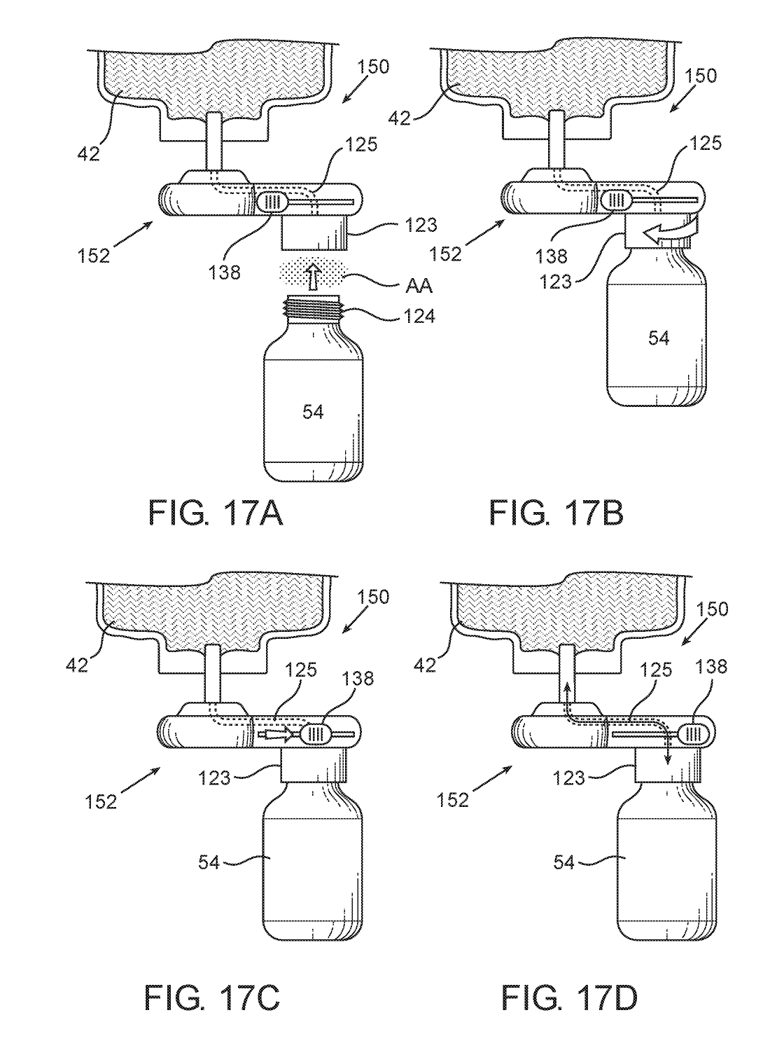

FIGS. 17A-17D demonstrate system 150 which further include port 123 which is internally threaded and can engage with thread 124 as an engagement mechanism between vessel 54 and housing 152. In this system, the vessel 54, once engaged with housing 152, is stationary and disposed in a longitudinal axis being different than the longitudinal axis of vessel 42. Vessels 42 and 54 are hence disposed such that their axes lie in different parallel longitudinal planes. FIG. 17A demonstrates vessel 54 and system 150 when spaced apart and AA surround thereof. FIG. 17B shows vessel 54 when engaging system 150 via a thread 124. In FIG. 17C a wiping member (not shown) disposed within the housing 152 actuated by tab 138 is operable to decontaminate a surface of vessel 54. FIG. 17D shows a fluid passageway formed by virtue of channel 125 disposed in the interior of housing 152. In this embodiment the wiping member (not shown) wipes off and decontaminates the surface of one vessel 54.

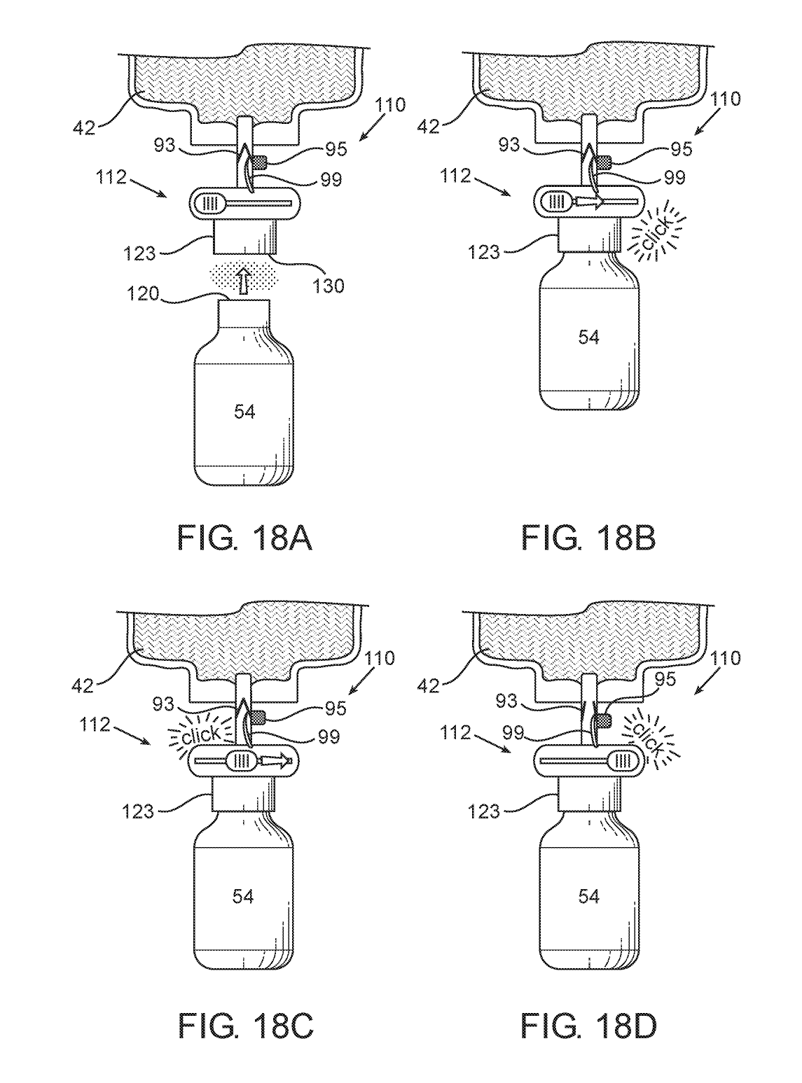

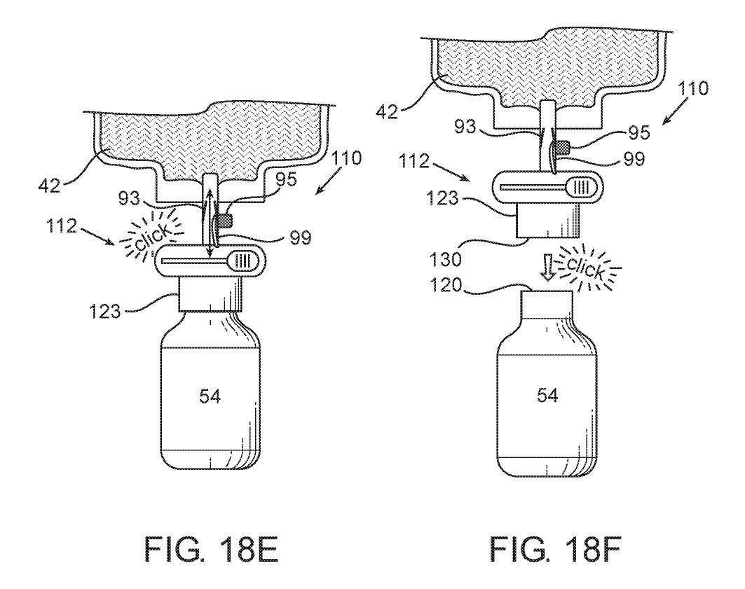

Reference is now made to FIGS. 18A-18F which illustrate system 110, wherein a click sound or any other sound may be heard at about the time or at the time of engagement of the vessel 54 with housing 112 (FIG. 18B) and/or at about the time or at the time of wiping the surface 120 of vessel 54 by wiping member (FIG. 18C) and/or at about the time or at the time of opening valve 93 (FIG. 18D), and/or at about the time, or the time of fluid passageway between vessels 42 and 54 (FIG. 18E) and/or at about the time, or the time of detachment of vessel 54 from the housing 112 (FIG. 18F).

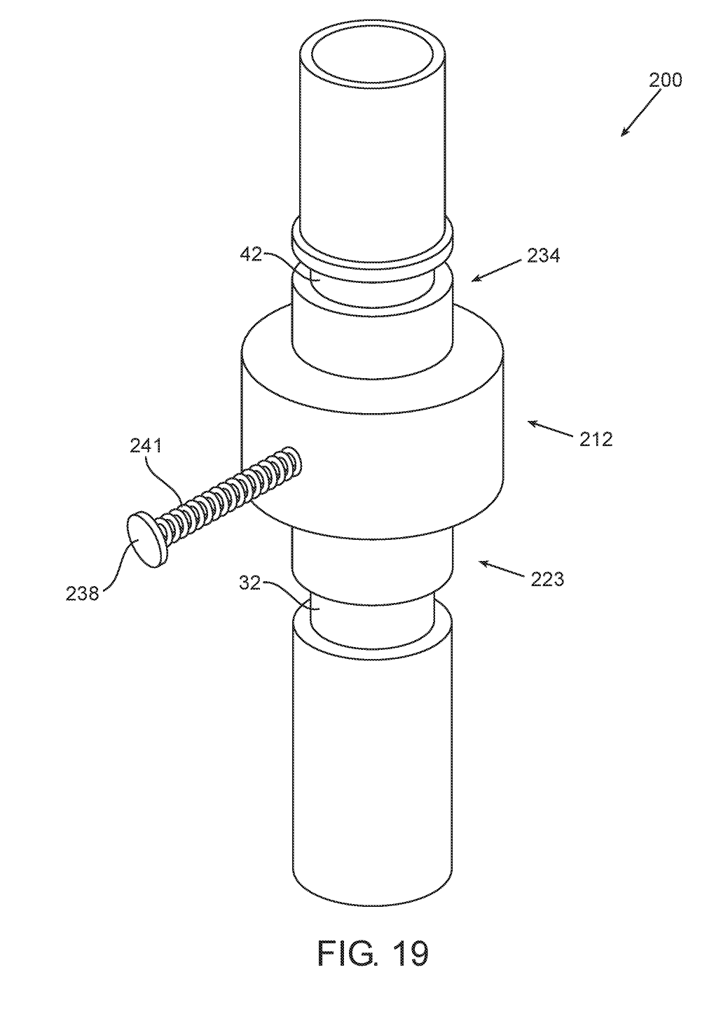

Referring to FIG. 19, a perspective view of yet a further exemplary system 200 is shown. System 200 includes a housing 212 with top port 234 allowing engagement with vessel 42 and bottom port 223 allowing engagement with vessel 32. A wiping member is disposed within the housing (not shown). The system includes a wiping member actuator in the form of handle 238 and spring 241.

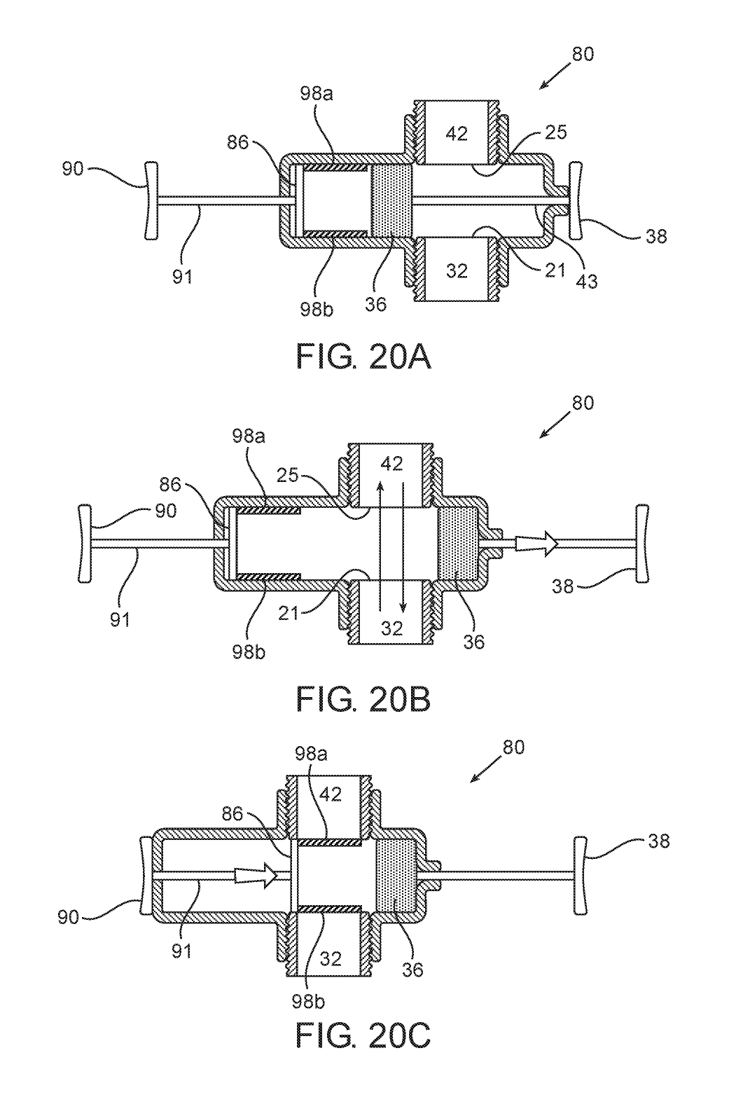

Reference is now made to FIGS. 20A-20C, an alternative embodiment of system 80 is shown. Here, the capping mechanism 98 of system 80 additionally or alternatively includes a capping actuator comprising a capping actuator handle 90 attached to capping actuator rod 91. Rod 91 is further attached vertically to rod pusher 86. FIG. 20A shows wiping member 36 when disposed in an initial position connected to rod 43 which is connected to rod handle 38. FIG. 20B shows wiping member 36 following decontamination of surfaces 21 and 25 of vessels 32 and 42, respectively. Wiping member 36 moves from the left side of the housing to the right side of the housing via a pulling of the rod handle 38. FIG. 20B shows the wiping member 36 disposed in an end position. FIG. 20C shows capping actuator after moving, optionally by a push sliding motion, top caps 98a and 98b to reseal vessels 42 and 32. Resealing of vessels 32 and 42 occurs within the decontaminated or sterilized housing.

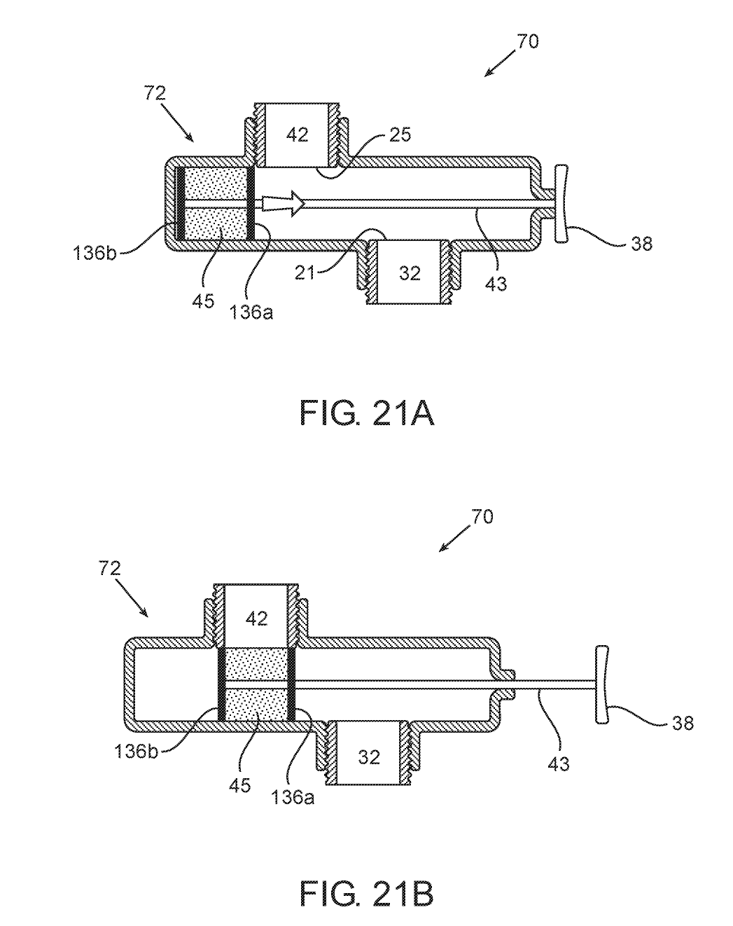

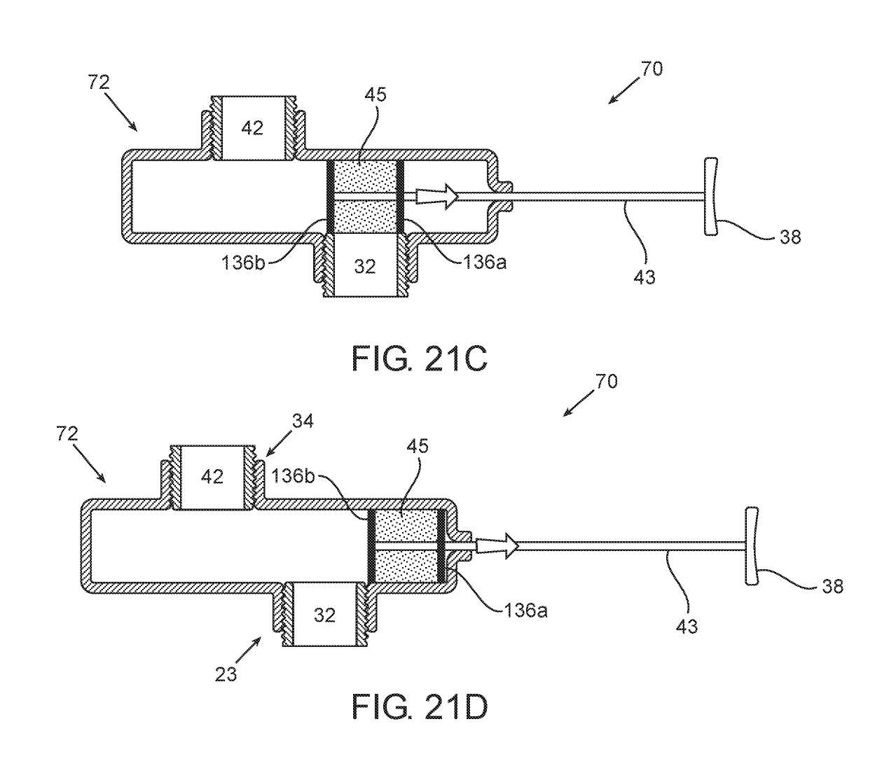

Reference is now made to FIGS. 21A-21D, an alternative embodiment of system 70 is shown. Here, sterilizing substance 45 replaces wiping member 36 and is used to decontaminate the surfaces 21 and 25 of vessels 32 and 42, respectively. It is to be noted that sterilizing substance 45, although shown with respect to system 70, can be provided along with or without a wiping member of any of the herein disclosed systems. Sterilizing substance 45 may be a liquid such as ethanol (e.g., ethanol 70%), isopropyl alcohol (e.g., isopropyl alcohol 70% or greater concentration), hydrogen peroxide (e.g., hydrogen peroxide 0.25% or greater concentration) or a gas. Sterilizing substance 45 may be enclosed between two wiping members 136a and 136b. The sterilizing wiping members 136a and 136b may be actuated to slide or move within housing 72 by actuator having handle 38 attached to rod 43. FIG. 21A demonstrate the wiping members 136a and 136b when disposed in an initial position. At FIG. 21B, the wiping members 136a and 136b and sterilizing substance 45 forcibly actuated to move or slide along housing 72 by handle 38 and rod 43, thereby decontaminating surface 25 of vessel 42. At FIG. 21C, the wiping members 136a and 136b and sterilizing substance 45 are shown when wiping off surface 21 of vessel 32 and At FIG. 21D the wiping members 136a and 136b and sterilizing substance 45 are shown at an end position.

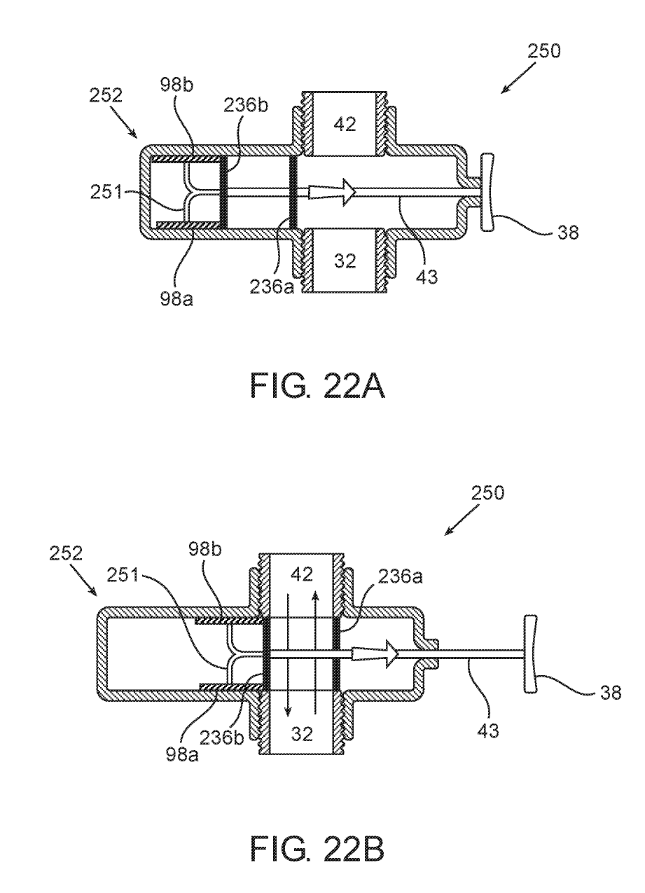

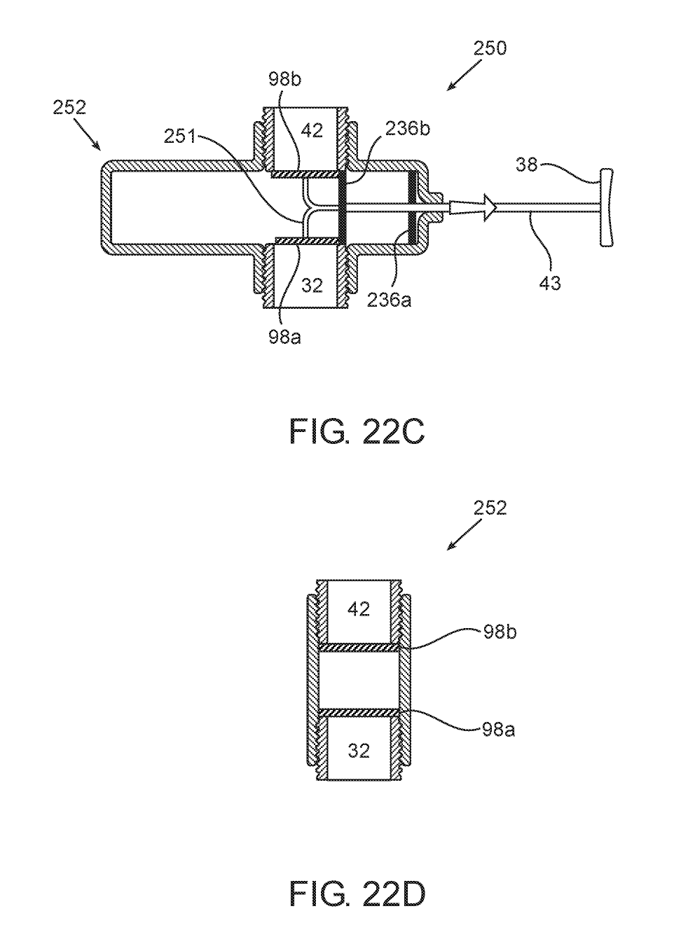

Referring now to FIGS. 22A-22C, a further exemplary system 250 is shown. System 250 is similar to system 80 and includes a capping mechanism including top cap 98a and bottom cap 98b. In this system top cap 98a and bottom cap 98b are being manipulated by rod 43 which is connected to an extension 251. Extension 251 is divided to attach top cap 98a and bottom cap 98b. A further optional embodiment of system 250 is a double wiper that includes two wiping members being wiping members 236a and 236b. The wiping members 236a and 236b are spaced apart in a manner allowing a fluid passageway between the wiping members 236a and 236b when a first wiping member 236a and a second wiping member 236b longitudinally aligned at opposing side walls of port of vessels 32 and 42. Such configuration provides wiping off vessels 32 and 42 by first wiping member 236a, forming a fluid passageway between vessels 32 and 42 (FIG. 22B) and thereafter wiping off again with wiping member 236b the vessels from any fluid residuals prior to recapping the vessels by top cap 98a and bottom cap 98b. FIG. 22D shows a side cut view of top cap 98a and bottom cap 98b resealing within the sterile housing 252 vessels 32 and 42 respectively.

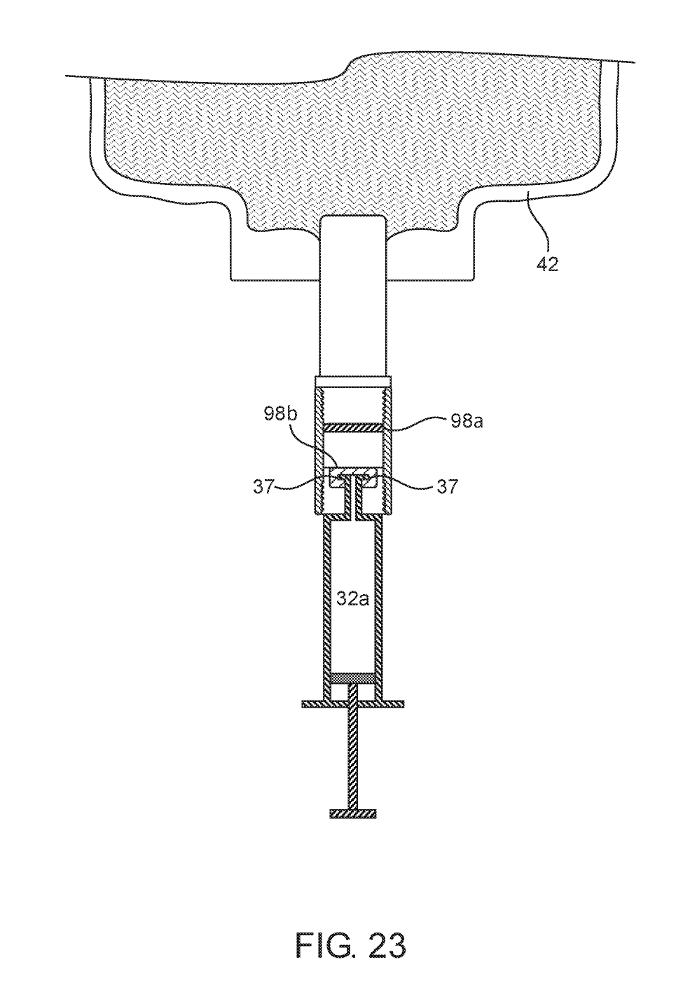

Referring now to FIG. 23, a side view of system 250 is shown whereby a syringe 32a is resealed within the housing by a displaceable member 98 (labeled on the left side of the housing), wherein the displaceable member 98 is a cap, cover, or any other type of resealing member. The displaceable member 98 and/or 98a engages the wings 37 of the syringe 32a within the housing 252.

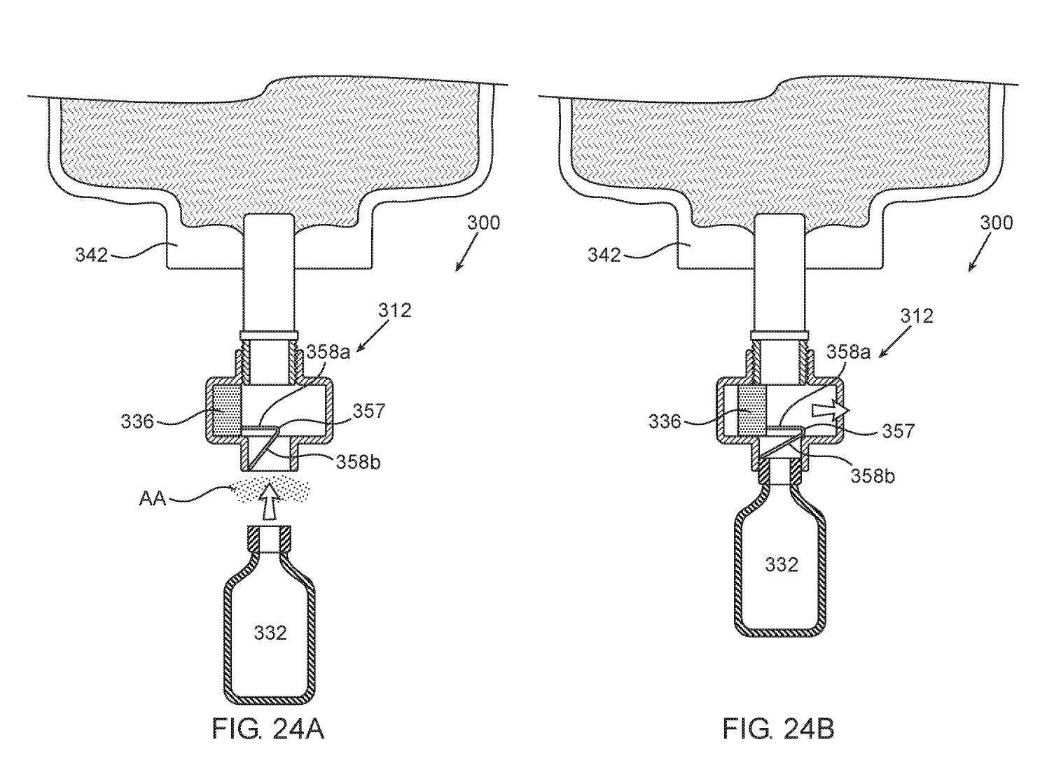

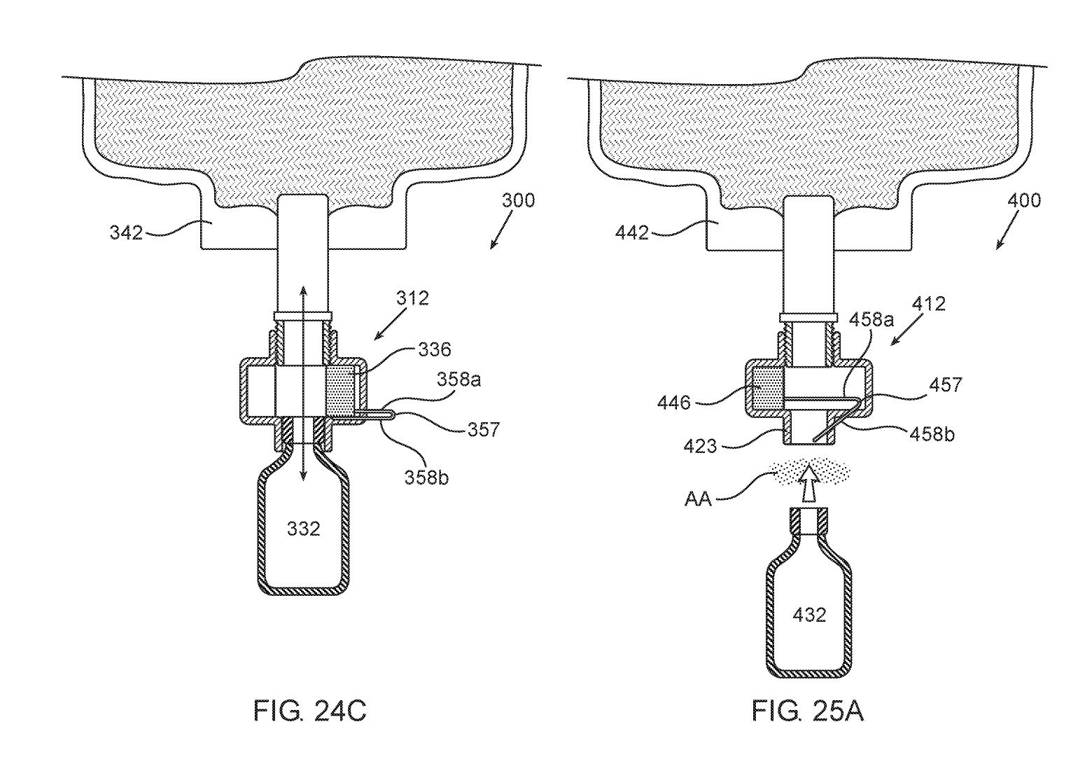

Referring now to FIGS. 24A-24C, which illustrate yet another embodiment of the invention, herein presented as system 300 that includes a pushing mechanism for actuating movement of the wiping member. In FIG. 24A, the vessel 332 is shown prior to an engagement with housing 312. Upon pushing vessel 332 into housing 312, a wiping member actuator in the form of resilient string 357 is actuated to effect movement of wiping member 336 across the housing 312. The string 357 may be bended having two arms, i.e., a first arm 358a and a second arm 358b. First arm 358a is attached to wiping member 336 and a second arm 358b is initially supported by a side wall of the housing 312 such to hold the wiping member 336 in an initial position in the housing 312. When vessel 332 is pushed into housing 312, the second arm 358b of resilient string 357 is pushed upwardly to release holding of the wiping member and allow movement thereof to an end position (FIGS. 24B-24C). The movement of the wiping member within the housing allows the wiping and decontamination effect of the wiping member 336.

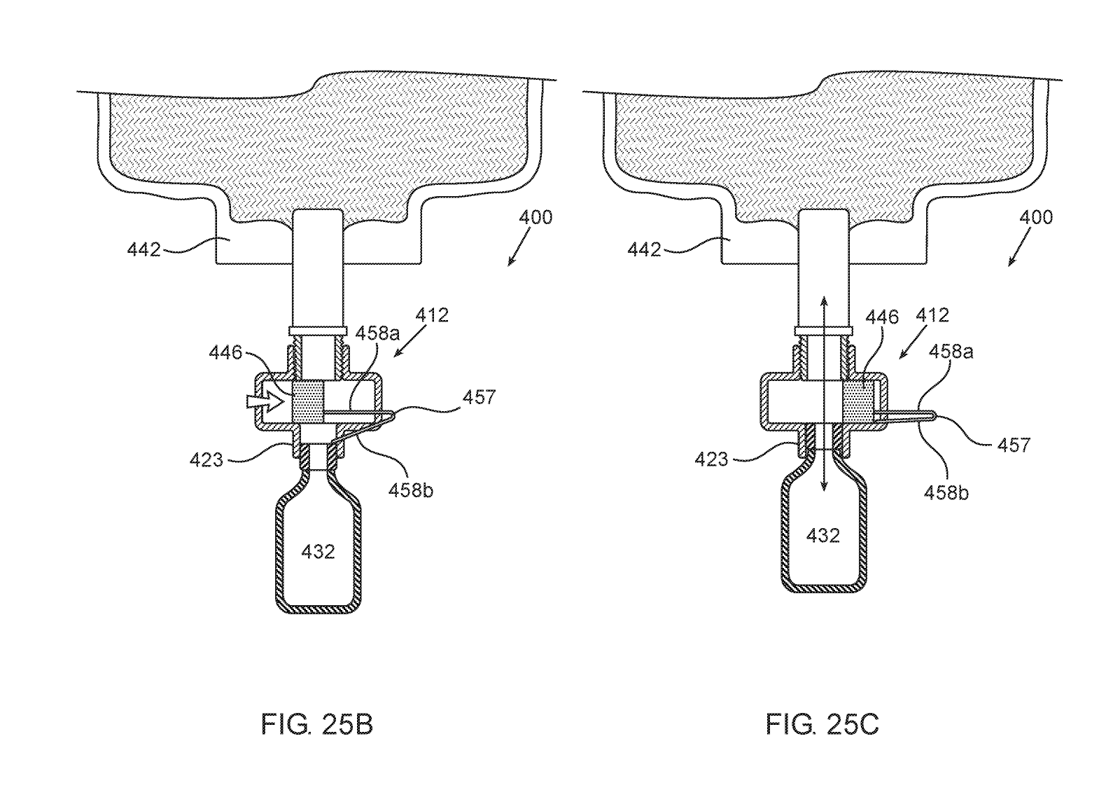

In FIGS. 25A-25C, yet another exemplary system 400 with a push mechanism for actuating the wiping member movement is shown. Here, a first arm 458a of the resilient string 457 is made longer and extends horizontally along the housing 412. Second arm 458b extends outwardly from an opening in a wall of the housing 412 and enters the housing via port 423 such that an end of the second arm 458b enters into the port 423 of the housing 412. Similarly, to system 300, when vessel 432 is pushed inwardly into housing 412, the string 457 is contracted and/or bended such that second arm 458b is pushed into a lumen of the housing 412 and releases holding of the wiping member, allowing movement thereof along housing 412 (FIGS. 25B-25C). The movement of the wiping member 446 along housing 412 may involve at least partial exit of string 457 from housing 412 (FIG. 25C). String 457 may be made of (but not limited to) a plastic material, a metal alloy, a flexible plastic, a flexible metal allow, a rubber material, and combinations thereof.

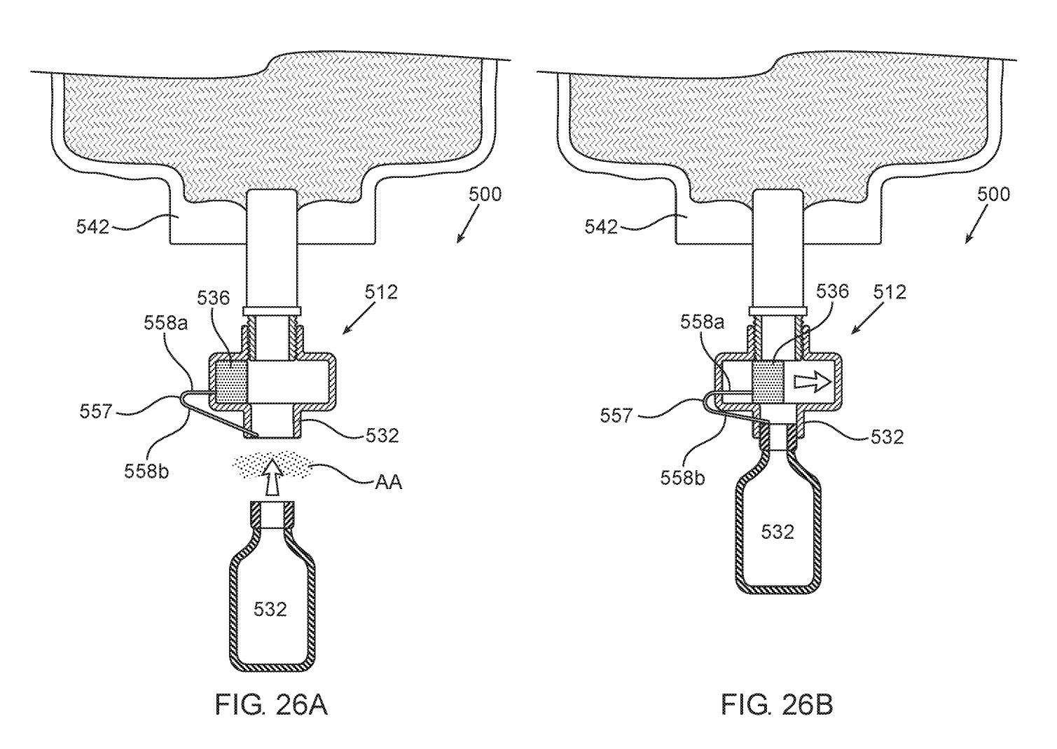

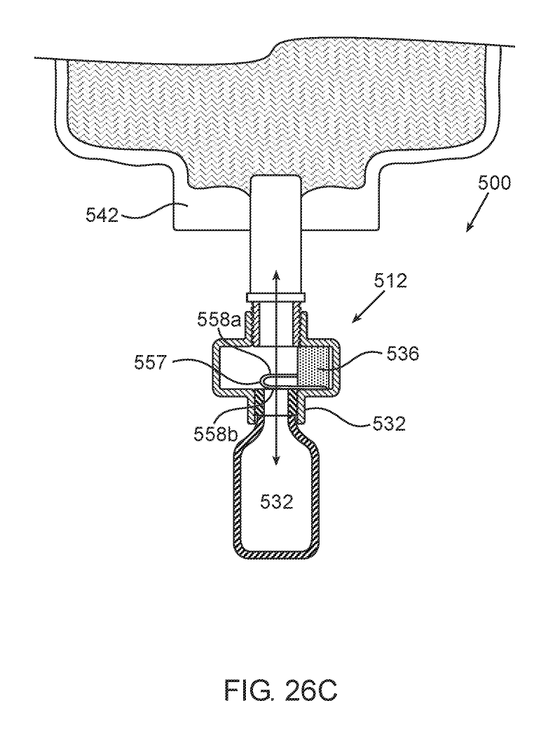

FIGS. 26A-26C illustrate a system 500 with a push mechanism for allowing movement of the wiping member 536. The string 557 is located initially outside housing 512. Here, first arm 558a of resilient string 557 extends from an attachment with wiping member 536 and together with second arm 558b located outside of housing 512. Second arm 558b or a portion thereof is located and protrudes into port 532 (of housing 512) such that when vessel 532 (shown as a vial container) enters the port 532 (of housing 512), the string 557 is bended and releases holding of wiping member 536, allowing movement thereof within housing 512. Other resealing engagement mechanisms disposed inside a sterile non-contaminated compartment of the housing are contemplated such as thread, luer, snap-on, slide-on, ratchet teeth, etc. and combinations thereof (not shown). The resealing engagement mechanisms may be configured to seal and/or reseal an aperture/opening/port of a vessel, wherein the vessel may be a syringe, a container, a bottle, a vial, an infusion line, or a connector.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for decontaminating a surface of at least one vessel, the device comprising:
a housing configured to allow an engagement with the at least one vessel;
a wiping member disposed within the housing; and
a wiping member actuator configured to maneuver movement of the wiping member along the housing upon a pushing motion exerted by the engagement of the at least one vessel with the housing, wherein the movement of the wiping member along the housing decontaminates the surface of the at least one vessel.

2. The device of claim 1, wherein the actuator is selected from a group consisting of a lever, a gear, a spring, a mechanical actuator, an electrical actuator, a string, a clamp, a pin and combinations thereof.

3. The device of claim 1, wherein the actuator is connected to the wiping member and configured to forcibly move the wiping member when the at least one vessel engages the housing.

4. The device of claim 1, wherein the actuator moves the wiping member via a pushing, a pulling and/or a sliding motion of the wiping member.

5. The device of claim 1, wherein the actuator extends through an opening in a wall of the housing.

6. The device of claim 5, wherein an airtight seal is maintained between the actuator and the opening in the wall.

7. The device of claim 1, wherein the actuator disposed within the housing, outside the housing or both.

8. The device of claim 1, wherein the pushing motion exerted by the engagement of the vessel with the housing transfers a force to the actuator to thereby allow the movement of the wiping member along the housing.

9. The device of claim 1, wherein the actuator holds the wiping member in an initial position and allows movement of the wiping member to an end position following pushing the vessel into a port of the housing.

10. The device of claim 1, wherein the actuator is a resilient string connected at one end thereof to the wiping member and having a second end or portion that protrudes into a lumen of a vessel's port within the housing.

11. The device of claim 10, wherein the string holds the wiping member in an initial position and wherein pushing a vessel into a port of the housing pushes said second end or portion of the string, thereby releasing holding of the wiping member in the initial position and allowing movement thereof to an end position.

12. The device of claim 1, which is configured to allow a fluid passageway between two or more vessels following the decontamination of the surfaces of the vessels.

13. A method of decontaminating a surface of at least one vessel, the method comprising:
providing a decontamination device having a housing, a wiping member disposed within the housing and one or more ports for receiving one or more vessels, wherein the wiping member disposed within the housing and configured to move within the housing;
providing at least one vessel having a surface;
connecting the decontamination device to the at least one vessel, by pushing the at least one vessel into the port of the decontamination device; and
moving the wiping member within the housing, thus, decontaminating the surface of the at least one vessel.

14. The method of claim 13, wherein the movement of the wiping member is exerted by the pushing motion of the at least one vessel into the port of the decontamination device.

15. The method of claim 13, wherein the device further comprises an actuator configured to move the wiping member across the housing following pushing the at least one vessel into the port of the decontamination device.

16. The method of claim 15, wherein the actuator is selected from a group consisting of a lever, a gear, a spring, a mechanical actuator, an electrical actuator, a string, a clamp, a pin and combinations thereof.

17. The method of claim 15, wherein the actuator is connected to the wiping member at one end thereof and having a second end or portion that protrudes into a lumen of the port such that when the vessel is pushed into the decontamination device, the second end or portion transfers the push forces to the actuator to thereby exert movement of the wiping member across the housing.

18. The method of claim 15, wherein the actuator moves the wiping member via a pushing, a pulling and/or a sliding motion of the wiping member.

19. The method of claim 13, wherein moving the wiping member across the housing forms a chamber/compartment/housing that is sterile and further forms a conduit where a fluid communication may be established between the at least one vessel and the decontamination device.

20. The method of claim 13, further comprising engaging the at least one vessel to the decontamination device via an engagement mechanism.

21. The method of claim 20, wherein the engagement mechanism is selected from a group consisting of a thread mechanism, a luer mechanism, a ratchet teeth/retention member mechanism, an adhesive mechanism, and combinations thereof.
